(19) United States
(12) Patent Application Publication
(10) Pub. No.: US 2006/0223841 A1
(43) Pub. Date: Oct. 5, 2006

(54) Stable pharmaceutical compositions of desloratadine and processes for preparation of polymorphic forms of desloratadine

(76) Inventors: Zoltan G. Toth, Debrecen (HU); Piroska Kovacs, Debrecen (HU); Csaba Peto, Debrecen (HU); Adrienne Kovacsne-Mezei, Debrecen (HU)

Correspondence Address: KENYON & KENYON LLP, ONE BROADWAY, NEW YORK, NY, 10004, US

(21) Appl. No.: 11/447,336

(22) Filed: Jun. 5, 2006

Related U.S. Application Data
(62) Division of application No. 11/283,276, filed on Nov. 17, 2005.
Application No. 11/283,276 is a continuation-in-part of application No. 10/800,290, filed on Mar. 12, 2004.
(60) Provisional application No. 60/526,339, filed on Dec. 1, 2003.
(60) Provisional application No. 60/516,904, filed on Nov. 3, 2003.
(60) Provisional application No. 60/515,354, filed on Oct. 28, 2003.
(60) Provisional application No. 60/454,299, filed on Mar. 12, 2003.

(51) Int. Cl. A61K 31/473; C07D 221/02

(52) U.S. Cl. 514/290; 546/93

(57) ABSTRACT

Provided are stable pharmaceutical compositions of desloratadine. Also provided are processes for preparing polymorphs of desloratadine

### PRIORITY

This application is a divisional application of U.S. application Ser. No. 11/283,276, filed Nov. 17, 2005, which is a continuation-in-part of U.S. application Ser. No. 10/800,290, filed Mar. 12, 2004 and claims the priority of provisional application Ser. No. 60/526,339, filed Dec. 1, 2003, provisional application Ser. No. 60/516,904, filed Nov. 3, 2003, provisional application Ser. No. 60/515,354, filed Oct. 28, 2003, and provisional application Ser. No. 60/454,299, filed Mar. 12, 2003. The contents of each of these applications are incorporated herein by reference.

### FIELD OF THE INVENTION

The present invention relates to the solid state chemistry of desloratadine and stable pharmaceutical compositions comprising desloratadine.

### BACKGROUND OF THE INVENTION

Desloratadine, known as 8-chloro-6,11-dihydro-11-(4-piperidylidene)-5H-benzo[5,6] cyclohepta[1,2-b]pyridine, has the following structure: 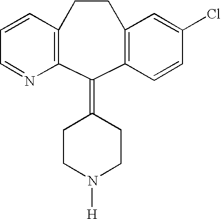
and is disclosed in U.S. Pat. No. 4,659,716. Desloratadine is currently marketed as Clarinex® in the United States. Clarinex is prescribed as an antihistamine for prevention or treatment of allergenic reactions, which may result in symptoms such as sneezing, itchy eyes and hives. The '716 patent discloses methods for preparing and administering desloratadine and its pharmaceutically acceptable salts, and is incorporated herein by reference. See also U.S. Pat. No. 4,282,233, incorporated herein by reference, which discloses loratadine.

The present invention relates to the solid state physical properties of desloratadine. These properties can be influenced by controlling the conditions under which desloratadine is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetry (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

In Example V, the '716 patent prepares desloratadine in the solid state and discloses: “Extract the organic material with chloroform, wash with water and remove the solvent. Triturate the residue with hexane. Recrystallize from a large volume of hexane after charcoal decolorization to obtain the product, m.p. 151°-152° C.”

In Example VI, B, desloratadine is also prepared in the solid state: “The material is extracted several times with chloroform, the chloroform extracts washed with water and concentrated to dryness, and the residue triturated with petroleum ether or hexane to yield 11.5 grams (93%) m.p. 149°-151° C. After recrystallization from hexane, the product melts at 150°-151° C.” The starting material for Example VI, B, is an N-cyano compound prepared according to the disclosure in U.S. Pat. No. 3,326,924.

Both U.S. Pat. No. 4,282,233 and U.S. Pat. No. 3,326,924 are incorporated herein by reference, particularly for their disclosure of preparation of desloratadine.

U.S. Pat. No. 6,506,767 discloses two polymorphic forms of desloratadine, labelled Forms I and II (syn. form 1 and form 2). The XRPD peaks and the FTIR spectrum for the forms are also disclosed in the '767 patent.

The '767 patent discloses: “Surprisingly we discovered that certain alcoholic solvents, e.g., hexanol and methanol produced 100% polymorph form 1, but others, e.g., 3-methyl-1-butanol and cyclohexanol produced significant amounts of form 2. Chlorinated solvents, e.g., dichloromethane produced form 1 substantially free of form 2 but the compounds were discolored. Ether solvents such as dioxane produced form 1 substantially free of form 2 but other alkane ethers, e.g., di-isopropyl ether produced form 1 with significant amounts of form 2 and di-n-butyl ether favored formation of form 2. Ketones such as methyl isobutyl ketone produced crystalline polymorph form 1 essentially free of form 2 but methyl butyl ketone produced a 8:1 ratio of form 1 to form 2. Use of methyl isubutyl ketone is preferred to produce crystalline polymorph form 1 essentially free of form 2. Only ethyl acetate and di-n-butyl ether were found to produce crystalline polymorph form 2 substantially free of form 1. Use of di-n-butyl ether is preferred for producing crystalline form 2 substantially free of fom 1.”

The '767 patent, in Examples 1-3, prepares Form I by crystallization out of methyl isobutyl ketone, while in examples 4 and 5, prepares Form II by crystallization out of ethyl acetate and di-n-butyl ether, respectively.

The '767 patent also carried out stability tests on Polymorph Form I. According to the '767 patent, Form I was “subjected to stability testing at various temperatures (25, 30 and 40° C.) and relative humidities of 60%, 60% and 75%, respectively . . . No significant change (<1%) from initial sample % form 1 and related compounds was observed.”

The '767 patent warns against using polymorphic mixtures of desloraratadine for formulation. According to the '767 patent, “such a mixture could lead to production of a [desloratadine] which would exist as a variable mixture of variable composition (i.e., variable percent amounts of polymorphs) having variable physical properties, a situation unacceptable in view of stringent GMP requirements.”

The '767 patent is incorporated herein by reference in its entirety, and more particularly in respect to its characterization of the polymorphic forms, synthesis of the starting material and preparation of the various polymorphic forms.

There is a need in the art for additional processes for preparation of polymorphic forms of desloratadine and pharmaceutical compositions of desloratadine.

### SUMMARY OF THE INVENTION

In one aspect, the present invention provides a process for preparing crystalline desloratadine Form I comprising the steps of preparing a solution of desloratadine in a solvent selected from the group consisting of acetonitrile, di-methyl formamide, tetrahydrofuran and diethylcarbonate, wherein desloratadine Form I crystallizes out of the solution and recovering the desloratadine Form I.

In another aspect, the present invention provides a process for preparing crystalline desloratadine Form I comprising the steps of preparing a solution of desloratadine in a solvent selected from the group consisting of chloroform and ethyl acetate, combining the solution with an anti-solvent to precipitate desloratadine Form I and recovering desloratadine Form I.

In another aspect, the present invention provides a process for preparing crystalline desloratadine Form I comprising the step of preparing a solution of desloratadine in a C₁ to C₄ alcohol, combining the solution with water to precipitate desloratadine Form I and recovering desloratadine Form I.

In another aspect, the present invention provides a process for preparing crystalline desloratadine Form I comprising the steps of preparing a solution of desloratadine in isopropanol, wherein desloratadine Form I precipitates from the solution; and recovering the desloratadine Form I.

In another aspect, the present invention provides a process for preparing crystalline desloratadine Form II comprising the steps of melting desloratadine to obtain a molten material, cooling the molten material to obtain a solid and grinding the solid.

In another aspect, the present invention provides a process for preparing a mixture of crystalline desloratadine Form I and Form II comprising the step of grinding crystalline desloratadine Form I.

In another aspect, the present invention provides a process for preparing crystalline desloratadine Form II comprising the steps of preparing a solution of desloratadine in dimethyl carbonate, wherein desloratadine Form II precipitates from the solution and recovering the desloratadine.

In another aspect, the present invention provides a process for preparing crystalline desloratadine Form I comprising the steps of preparing a solution of desloratadine in i-butyl acetate, wherein Form I precipitates from the solution and recovering the precipitate.

In another aspect, the present invention provides a process for preparing crystalline desloratadine Form I comprising the steps of preparing a solution of desloratadine in a solvent selected from the group consisting of isopropanol and i-butanol, wherein desloratadine Form I precipitates from the solution and recovering the mixture.

In another aspect, the present invention provides a process for preparing a mixture of crystalline Form I and Form II of desloratadine comprising the step of drying desloratadine Form I crystals obtained by crystallization from a C₁ to a C₄ alcohol.

In another aspect, the present invention provides a process for making a mixture of crystalline desloratadine Form I and Form II comprising the steps of combining a solution of desloratadine in a suitable solvent with an anti-solvent containing seeds of both Form I and Form II of desloratadine to precipitate the mixture, and recovering the mixture.

In another aspect, the present invention provides a process for preparing a mixture of desloratadine crystalline Forms I and II containing at least about 25% of both of the Forms comprising the steps of preparing a solution of desloratadine in a solvent selected from the group consisting of ethyl acetate and iso-butyl acetate, in a mixture with about 3% to about 20% C₁ to C₄ alcohol by volume, wherein the mixture of Form I and II precipitates from the solution and recovering the mixture.

In another aspect, the present invention provides a process for preparing a mixture of crystalline desloratadine Form I and II comprising the steps of preparing a solution of desloratadine in iso-butyl acetate, combining the solution with a C₅ to C₁₂ aromatic hydrocarbon to precipitate the mixture, wherein the combining may be carried out before, after or during crystallization and recovering the mixture.

In another aspect, the present invention provides a process for preparing a mixture of crystalline desloratadine Form I and II comprising the steps of preparing a solution of desloratadine in iso-butyl acetate, combining the solution with iso-butyl acetate at a temperature lower than the solution to crystallize the mixture and recovering the mixture.

In another aspect, the present invention provides a process for preparing a mixture of crystalline desloratadine Form I and Form II comprising the steps of preparing a solution of desloratadine in ethyl acetate, seeding the solution with a mixture of Form I and Form II, combining the solution with a C₅ to C₁₂ saturated hydrocarbon, wherein the combining may be carried out before, after or during crystallization and recovering the mixture of desloratadine Form I and II.

In another aspect, the present invention provides a process for preparing a mixture of crystalline desloratadine Form I and Form II comprising the steps of preparing a solution of desloratadine in 2-propanol and toluene, wherein the mixture of Forms I and II precipitates from the solution and recovering the mixture.

In another aspect, the present invention provides a process for preparing a mixture of Form I and Form II, comprising the steps of providing a first solution of desloratadine in toluene, evaporating the toluene to obtain a residue, dissolving the residue in a mixture of toluene and a C₁ to C₄ alcohol to obtain a second solution, cooling the second solution to obtain a slurry, combining the slurry with a C₅ to C₁₂ saturated hydrocarbon to precipitate the mixture and recovering the mixture.

In another aspect, the present invention provides a process for preparing a mixture of desloratadine Form I and Form II comprising the steps of combining desloratadine acetate, toluene and KOH to obtain a reaction mixture, heating the mixture, whereby two phases are obtained, separating the phases, concentrating the separated organic phase, dissolving the obtained concentrate in a toluene-2-propanol mixture containing less than about 20% 2-propanol by volume, cooling the solution to obtain a slurry, combining the slurry with cold n-heptane and recovering mixture of desloratadine forms I and II.

In another aspect, the present invention provides a process for preparing crystalline desloratadine Form II comprising the steps of crystallizing desloratadine from toluene, and recovering the crystalline form.

The present invention also provides a pharmaceutical composition of desloratadine comprising of a mixture of crystalline form desloratadine I and II in a weight to weight ratio of about 25% to about 75% of either form to the other and a pharmaceutically acceptable excipient. Some the ratio is approximately 50%.

In another aspect, the present invention provides for a pharmaceutical composition of desloratadine comprising of crystalline form desloratadine I and II in a weight to weight ratio of about 20% to about 40% of Form II and a pharmaceutically acceptable excipient.

In another aspect, the present invention provides for a pharmaceutical composition of desloratadine prepared by a process comprising the steps of preparing a mixture of crystalline form desloratadine I and II in a weight to weight ratio of about 20% to about 40% Form II (Or up to 75% each) to Form I and combining the mixture with a pharmaceutically acceptable excipient to obtain a pharmaceutical composition.

In another aspect, the present invention provides for a stable mixture of crystalline form desloratadine I and II in a weight to weight ratio of about 25% to about 75% of either form to the other.

In another aspect, the present invention provides for a stable mixture of crystalline form desloratadine in a weight to weight ratio of from about 20-40% Form II to about 60-80% Form I.

In another aspect the present invention provides a stable mixture, and pharmaceutical compositions thereof, of crystalline form desloratadine I and II in a weight to weight ratio of about 25% to about 75% of either form, prepared by combining desloratadine salt, toluene and a base to obtain a reaction mixture; heating the mixture, whereby two phases are obtained; separating the phases; concentrating the separated organic phase; dissolving the obtained concentrate in a toluene-2-propanol mixture containing less than about 20% 2-propanol by volume; cooling the solution to obtain a slurry; combining the slurry with cold n-heptane; and recovering mixture of desloratadine forms I and II.

In one aspect the present invention provides a process for preparing a mixture of crystalline Form I and Form II of desloratadine comprising combining a solution of desloratadine in an organic solvent selected from a C₂ to C₅ alkyl acetate, butanol, isobutanol, toluene and chloroform with a C₅ to C₁₂ hydrocarbon to precipitate the mixture, recovering the mixture, wherein before combining, the solution has a temperature that is at least about 40° C. higher than the hydrocarbon. Preferably, the mixture has a residual level of any one of the organic solvent and C₅ to C₁₂ hydrocarbon that is less than about 5000 ppm. Preferably, the mixture comprises about 35-82% desloratadine Form I and about 18-65% desloratadine Form II.

In another aspect the present invention provides a mixture of crystalline Form I and Form II of desloratadine, containing about 50 ppm to about 4000 ppm of any one of isobutyl acetate, n-heptane, n-hexane, ethyl acetate, butanol, isobutanol, toluene, chloroform and combinations thereof. Preferably, the mixture comprises about 35-82% desloratadine Form I and about 18-65% desloratadine Form II.

In yet another aspect the present invention provides a pharmaceutical formulation comprising the mixture of crystalline Form I and Form II of desloratadine, containing about 50 ppm to about 4000 ppm of any one of isobutyl acetate, n-heptane, n-hexane, ethyl acetate, butanol, isobutanol, toluene, chloroform and combinations thereof.

### BRIEF DESCRIPTION OF THE FIGURES

FIG. 1 is a stability study of a polymorphic mixture of desloratadine.

FIG. 2 is a DSC thermogram of desloratadine Form II after grinding and sieving.

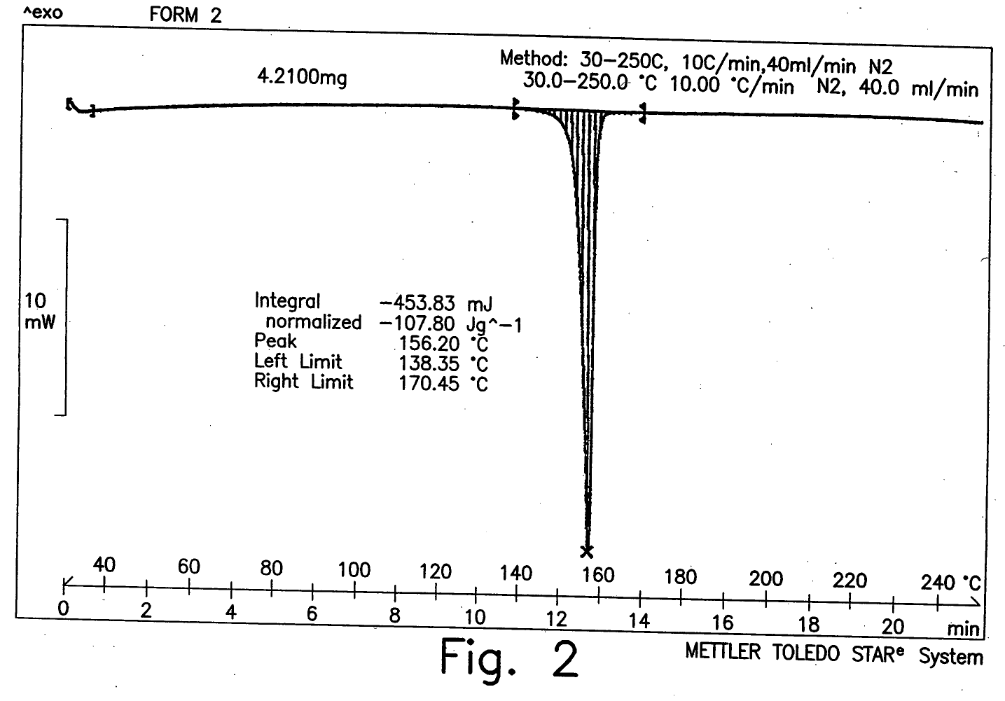

FIG. 3 is a DSC thermogram of desloratadine Form I after grinding and sieving.

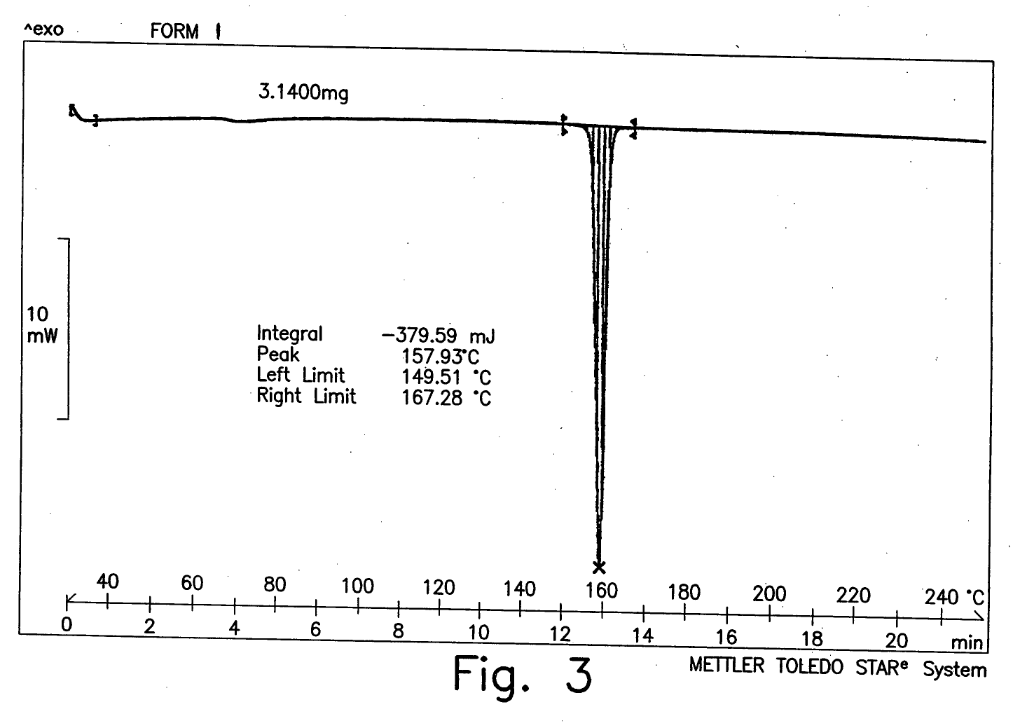

FIG. 4 is a DSC thermogram of a 25:75 mixture of Form I and Form II by weight.

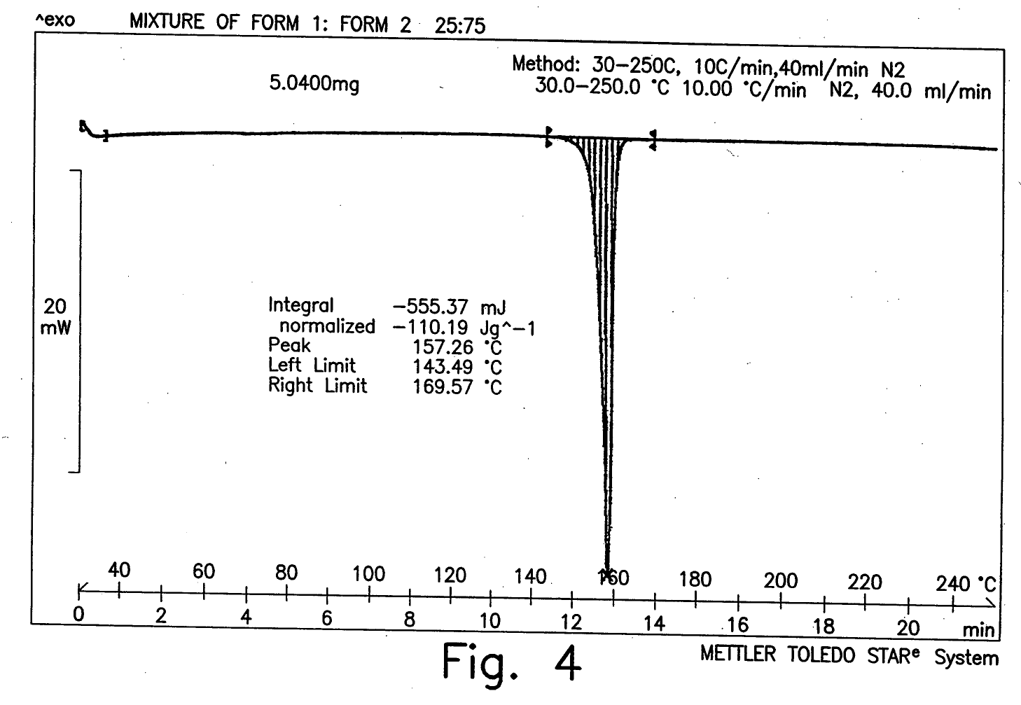

FIG. 5 is a DSC thermogram of a 50:50 mixture of Form I and Form II by weight.

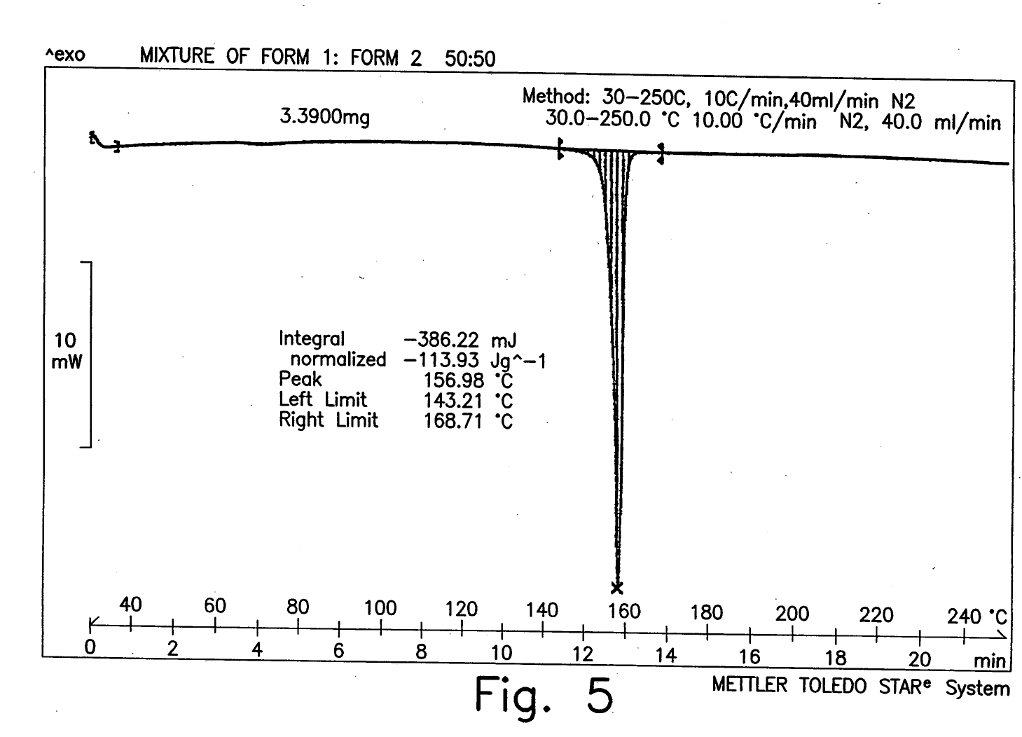

FIG. 6 is a DSC thermogram of a 75:25 mixture of Form I and Form II by weight.

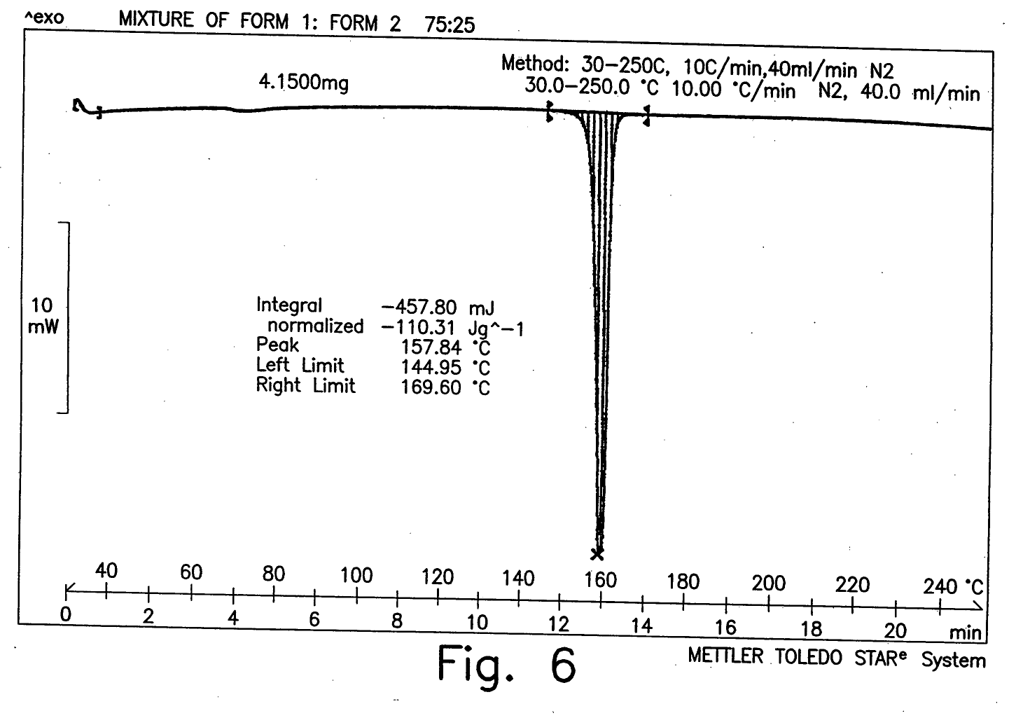

FIG. 7 is a DSC thermogram of a 84:16 mixture of Form I and Form II by weight.

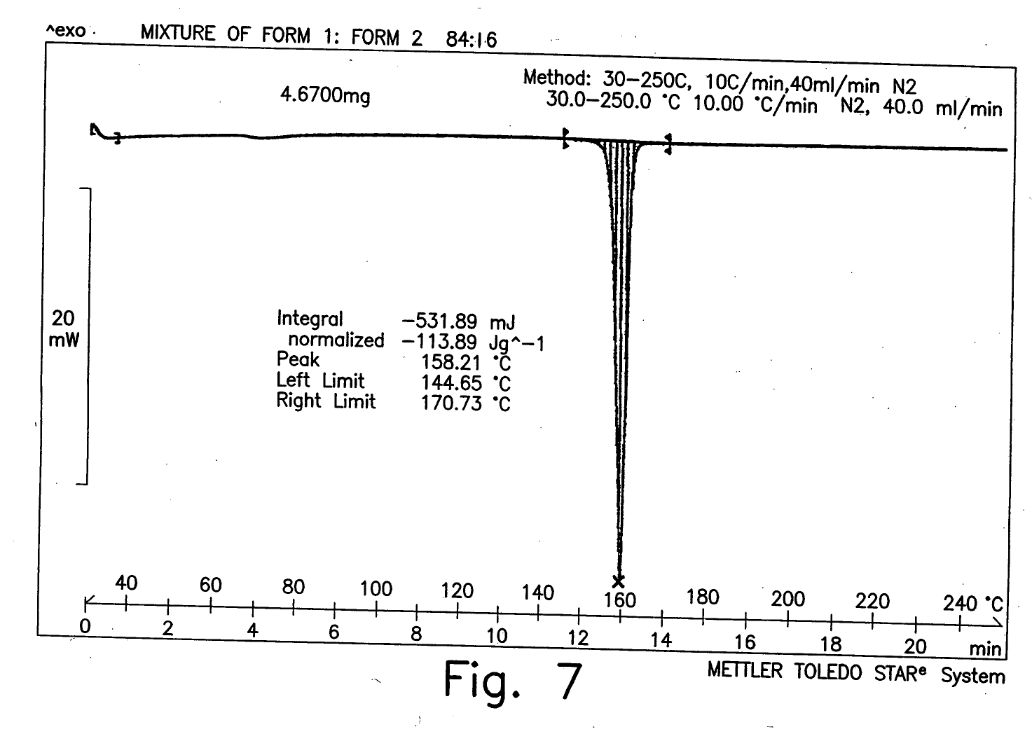

FIG. 8 is a comparison of X-ray powder diffraction patterns of desloratadine Form I and Form II, and various mixtures thereof.

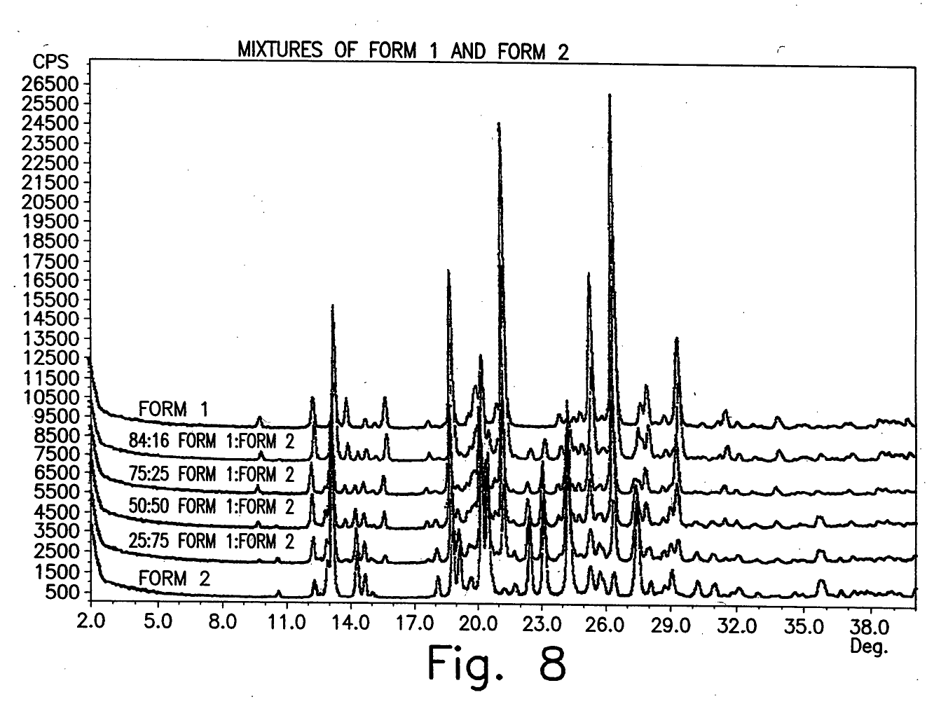

FIG. 9 is similar to FIG. 8, but illustrates the X-ray diffraction patterns after grinding.

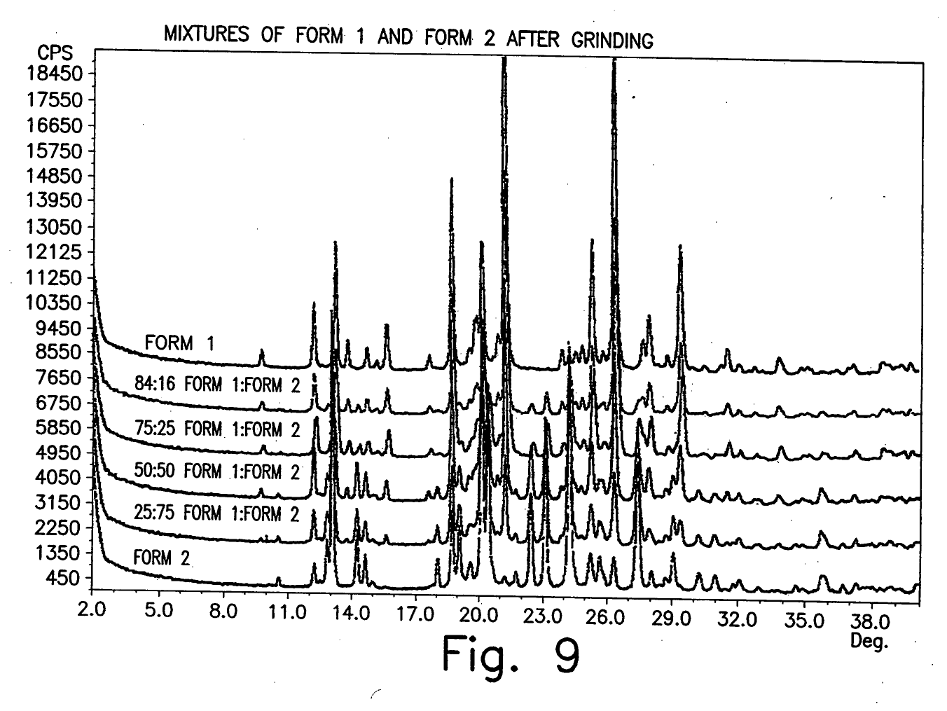

FIG. 10 is similar to FIG. 8, but illustrates the X-ray diffraction patterns after storage at 100% relative humidity.

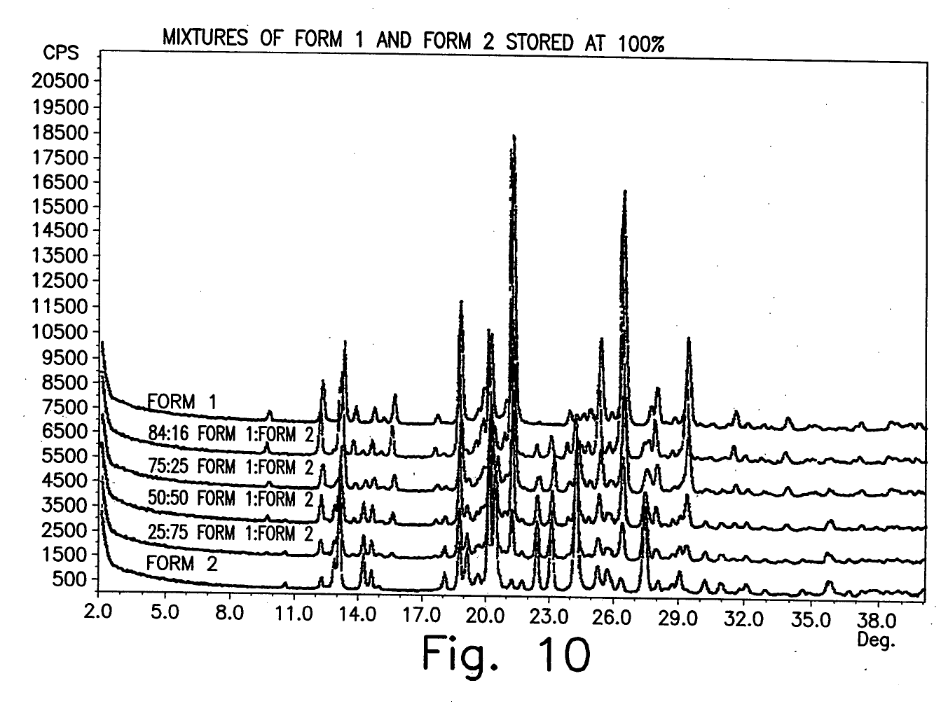

FIG. 11 is similar to FIG. 8, but illustrates the X-ray diffraction patterns after storage at 80% relative humidity.

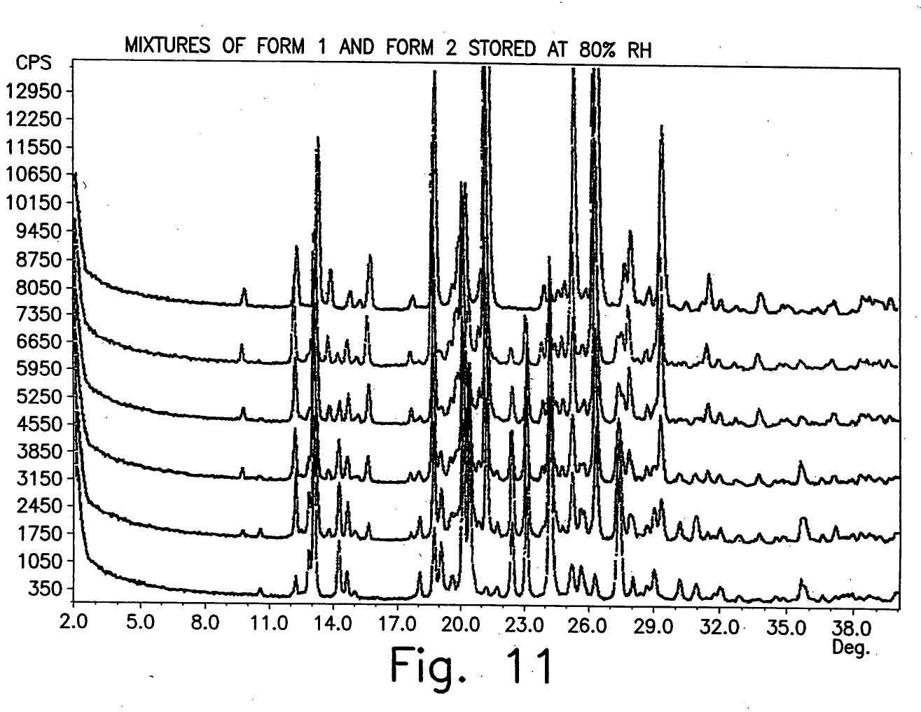

FIG. 12 is similar to FIG. 8, but illustrates the X-ray diffraction patterns after storage at 60% relative humidity.

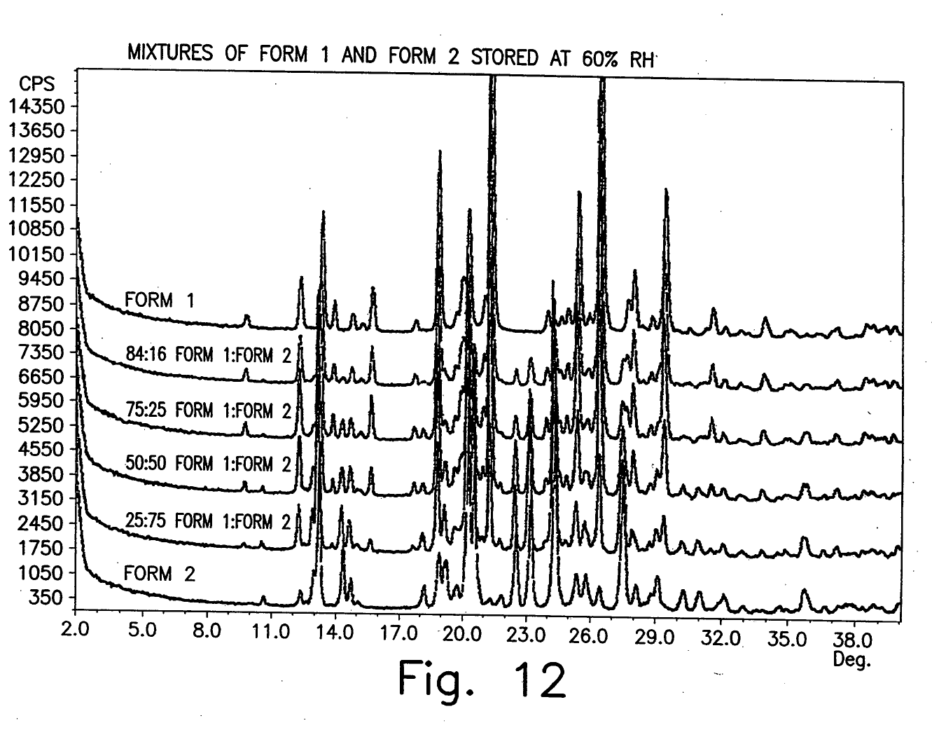

### DETAILED DESCRIPTION OF THE INVENTION

As used herein, the term “drying” refers to removal of solvent from a solid through application of heat.

As used herein, the term “C₅ to C₁₂ saturated hydrocarbon” refers to a straight/branched and/or cyclic/acyclic hydrocarbon. Preferred hydrocarbons are cyclohexane, cycloheptane, cyclohexane, n-heptane and n-hexane, with n-hexane and n-heptane being preferred. The terms “hexane” and “heptane” used hereinafter refer to n-hexane and n-heptane.

As used herein, the term “C₅ to C₁₂ aromatic hydrocarbon” refers to substituted and unsubstituted hydrocarbons having a phenyl group as their backbone. Preferred hydrocarbons include benzene, xylene and toluene, with toluene being more preferred.

As used herein, the term “C₃ to C₇ ester” refers to an ester having such number of carbons. Preferred esters include ethyl acetate.

As used herein, an “anti-solvent” is a liquid that when added to a solution of X in the solvent, induces precipitation of X. Precipitation of X is induced by the anti-solvent when addition of the anti-solvent causes X to precipitate from the solution more rapidly or to a greater extent than X precipitates from a solution containing an equal concentration of X in the same solvent when the solution is maintained under the same conditions for the same period of time but without adding the anti-solvent. Precipitation can be perceived visually as a clouding of the solution or formation of distinct particles of X suspended in the solution or collected at the bottom the vessel containing the solution.

The amount of Form I and Form II is expressed herein as a weight ratio relative to each other, i.e., (Form I or II)/Form I plus Form II×100%.

The present invention provides a process suitable for industrial scale preparation of formulations/compositions of desloratadine. Desloratadine may be crystallized as a mixture of polymorphs in such a way that the ratio between the polymorphs is consistent. As used herein, a “consistent ratio” (or consistent mixture) refers to a ratio of Form I compared to Form II (wt/wt) that is between a range of about ±10% (wt/wt) between lots, as measured by XRPD or FTIR.

In one embodiment, the pharmaceutical composition is prepared using a solution of desloratadine in toluene. The concentration of desloratadine is preferably at least about 15% by weight.

The desloratadine used may be obtained from loratadine, by hydrolysis of the carbamate, preferably under basic conditions. Loratadine itself may be prepared from N-methyl desloratadine by removing N-methyl group of N-methyl desloratadine by formation of the carbamate through reaction with a haloformate. The haloformate used may be an alkyl or aryl formate, with optional halogen substituted at first and/or second position of the formate, i.e., 2-chloroethyl-chloroformate. The carabmate may be prepared in an anhydrous C₅ to C₁₂ hydrocarbon, such as toluene. When N-methyl desloratadine is used as a stating material, loratadine may or may not be isolated in preparation of desloratadine.

The removal of the carbamate group of loratadine may be carried out with a base at elevated temperature. A preferred temperature is reflux temperature. A preferred base is an alkali metal or alkaline earth metal base such as potassium or sodium hydroxide. A preferred solvent is a C₁ to a C₄ alcohol such as 2-propanol.

A salt of desloratadine may be used as starting material, particularly since salt formation may be used to purify the starting material. A suitable salt is the acetate salt.

When starting from a salt, depending on the solubility of the salt, the salt may be suspended in toluene as to form a slurry. A base is then added to the slurry to obtain the free acid, which is readily soluble in toluene, and moves into solution. Suitable bases include those of alkali metal and alkaline earth metals such as potassium, sodium and calcium oxide/hydroxide/carbonate, preferably sodium or potassium hydroxide.

The base is preferably added as an aqueous solution to the toluene, where two phases form. An about a 2% to about 6% solution of sodium or potassium hydroxide, preferably about a 4% solution may be used. The slurry is preferably heated to increase the reaction rate, to for example a temperature of about 40 to about 70° C. The resulting two phase reaction system is preferably stirred at this temperature until complete dissolution.

The reaction results in neutralization of the salt, leading to solution of desloratadine free acid in toluene. After phase separation, such as by physical means with use of a separatory funnel, the toluene solution of desloratadine may be washed with distilled water at the same temperature to obtain more of the acid before discarding the aqueous phase.

In one embodiment, the resulting toluene solution is concentrated by vacuum or at atmospheric pressure (jacket: preferably about 55° C. to about 130° C.) to dryness, though it is theoretically possible to precipitate the acid by reducing the solubility of the solvent. The solid material is then dissolved in a mixture of toluene and 2-propanol, in the ratio of about 5:1 to about 15:1, more preferably about 9:1. The addition of relatively minor amounts of 2-propanol (anti-solvent) to toluene manipulates the ratio of Form I and II, and allows for a more facile crystallization. Without 2-propanol, substantially Form II is obtained rather than a mixture.

The mixture is preferably warmed to increase its solubility, such as to a temperature of about 50 to about 70° C., more preferably about 60° C. The warm solution is then preferably cooled to a temperature of about 10° C. to about 30° C., more preferably to about 20° C. The cooling may be carried out slowly, during a span of few hours. Cooling in about 4 hours is optimal. After cooling, the resulting slurry is then preferably stirred for a few hours, more preferably of about 5 to about 8 hours.

This slurry is preferably warmed again, to about 45 to 55° C., and dropped into cold n-heptane, preferably at about −5 to about +5° C. The precipitated solid material is then recovered preferably by filtration, and may be dried. Drying may be carried out at ambient or reduced pressure. In one embodiment, drying is carried our in a vacuum oven at about 25-35° C. overnight.

The desloratadine from the reaction may then be recovered as a polymorphic form. In a preferred embodiment, the reaction mixture is distributed between an organic phase and water, resulting in desloratadine moving to the organic phase. The process described above with toluene may then be used, where a solution of desloratadine in toluene is prepared.

The compositions of the present invention are substantially physically stable, i.e., substantially stable against polymorphic transformations. The stable compositions may be manufactured in accordance with the acceptable GMP requirements. The stability tests below show the relative stability of the two forms for at least about two months under accelerated conditions. The mixtures of physically stable and undergo less than about 10%, more preferably less than about 5% and most preferably less than about 3% polymorphic change per each polymorph after storage under accelerated ageing conditions (40° C. and 75% RH) for at least about 2 months. The mixtures also undergo less than about 10%, more preferably less than about 5% and most preferably less than about 1% polymorphic change in each polymorph when stored for at least about 2 months at room temperature and 60% RH. Additional stability may be imparted by formulating the desloratadine. The pharmaceutical formulation as a mixture may include stable mixtures of about 25% to about 75% weight/weight of one form compared to the other, such as about 25% of Form I, about 50% of Form I and about 75% of Form I, with the rest Form II. In one embodiment, about a 55-65% of Form I and about 35-45% of Form II mixture is used. In another embodiment about 20 to about 40% of Form II is used, more preferably about 24% to about 38%.

The term “GMP requirement” refers to consistency among batches. The physical properties concerning GMP requirements are stability and solubility. The dissolution rate was not tested due to very low solubility of desloratadine. Nevertheless, the dissolution rate of the stable mixture, when measured by the USP Paddle Method at 50-90 RPM in 900 mL water is preferably not less than about 80% by weight of the mixture released after 30 minutes. Preferably, the solubility of the bactches is within about a ±10%, more preferably within about ±5%, and most preferably within about ±1-3% compared to each other.

Stability of desloratadine at relative humidities of 60% 80% and 100% RH, stability under grinding, thermal stability/melting point in the DSC was monitored. The stable mixtures of 25:75, 50:50, 75:25, 84:16 (Form 1:Form 2) do not show any substantial change (Chemical: by degradation; Physical: by transformation to another polymorphic form) in the XRD pattern after exposure at 60%, 80%, 100% RH for one week. Also those stable mixtures do not show any substantial change in the XRD pattern after grinding for one minute; The sample is ground by hand in a mortar and pestle for about 1 minute. The separate polymorphs (Form I and Form II) were also monitored as a reference, and shown to be stable as well. The mixtures also show a substantial lack in chemical decomposition after storage at 100% humidity for one week and after grinding for 1 minute. This lack of decomposition is preferably undetectable by XRD and NMT 3%, more preferably NMT 2%, and most preferably NMT 3% by weight.

The physical properties of the two separate polymorphs (Form I and Form II) were compared to the physical properties of some mixtures (25:75, 50:50, 75:25, 84:16 Form I:Form II). It was discovered that polymorphic mixtures with different polymorphic compositions have practically invariable physical properties as compared to the separate polymorphs (Form I and Form II). Hence, even if there is polymorphic transformation, the thermal characteristics of the polymorphic mixture may remain substantially the same, which is ideal for formulation.

The thermal stability of the mixtures of polymorphs is comparable to that of the separate polymorphs. The melting temperatures of the stable mixtures, as determined in the DSC, is in the range of 157-158° C., while the separate polymorphs give in the DSC melting temperatures of 156° C. and 158° C. for Form II and Form I respectively. The similarity in melting points of the separate polymorphs and the stable mixtures indicates that the physical properties are not altered significantly. The DSC curves of the separate polymorphs also do not show any exotherm of decomposition at the temperature above the melting temperature, and also the stable mixtures do not show any event of decomposition above the melting temperature. This lack of decomposition indicates that the mixtures like the separate polymorphs are substantially thermally stable.

A particularly preferred range for the mixture of the present invention is about 20% to about 40% Form II compared to Form I.

The stable mixtures may be analysed by (FTIR) or X-Ray powder diffraction. Both techniques can be used to monitor polymorphic changes. X-Ray is reported in the literature for its capability to detect generally around 5% polymorphic impurities, but in many cases also to about 1% by weight. With FTIR however, the level of detection is not as accurate.

The present invention also provides various processes for preparing polymorphic Forms I and II of desloratadine, and mixtures thereof.

The mixtures of Form I and Form II for pharmaceutical formulations may be prepared by using a solution of desloratadine in a C₅ to C₁₂ aromatic hydrocarbons such as toluene. The concentration of desloratadine is preferably at least about 15% by weight. The solution is then combined with an anti-solvent, preferably a C₁ to C₄ alcohol such as isopropanol or methanol, more preferably in a ratio of about 7 to about 14% compared to the volume (v/v) of toluene. The resulting precipitate is then recovered by conventional techniques. Seeding to manipulate crystallization is optional.

In one embodiment, desloratadine so prepared is crystallized out from a solution of ethyl acetate by seeding with a mixture of Form I and Form II to obtain a solid, followed by addition of a C₅ to C₁₂ aromatic or saturated hydrocarbons such as heptane. This embodiment also encompasses use of hydrocarbon as an anti-solvent, where crystallization happens after addition of the hydrocarbon (Seeding may be carried out before, during or after adding the hydrocarbon). A solid may then be recovered by techniques known in the art such as filtration and dried, preferably at about vacuum (Pressure below about 50 mm Hg) and about room temperature. This embodiment produces from about a 4:1 to about a 1:3 mixture of Form I to Form II.

In another embodiment, desloratadine is crystallized out of a mixture of toluene and a C₁ to C₄ alcohol such as 2-propanol. See Example 28 and 29. In a preferred embodiment, a solution of desloratadine in heptane is concentrated and combined with 2-propanol, preferably the 2-propanol being less than 20% by volume. Crystallization may be carried out by heating the solution, preferably at a temperature above about 60° C. followed by addition of 2-propanol and cooling, preferably to a temperature below about 30° C. The crystals may be recovered by conventional techniques. The addition of relatively minor amounts of 2-propanol to toluene manipulates the ratio of Form I and II, and allows for a more facile crystallization. For example, when crystallization is carried out from toluene, substantially Form II is obtained, but addition of up to about 20% volume of a C₁ to C₄ alcohol as co-solvent allows for obtaining a mixture of the forms. This technique allows for carrying out much of the synthetic process in toluene, and yet obtain a mixture from toluene.

In one embodiment, a mixture of desloratadine Form I and Form II is precipitated out of a suitable solvent such as chloroform or ethyl acetate by addition of an anti-solvent. Desloratadine is dissolved to an organic solvent such as chloroform or ethyl acetate. Dissolution may be carried out by adding desloratadine to the solvent and heating the solvent to obtain a clear solution. A suitable anti-solvent is then added to precipitate the mixture. Examples of such anti-solvents include C₅ to C₁₂ saturated hydrocarbons, preferably saturated aliphatic hydrocarbons such as hexane and heptane, with hexane being more preferred. Another example of an anti-solvent is an ether whose alkyl radical groups connected to the oxygen atom are independently selected from the group consisting of methyl, ethyl, propyl, isopropyl, butyl, 1-butyl, 2-butyl and t-butyl. Most preferably, the ether is di-isopropyl ether. Preferred ethers are MTBE, di-isopropyl ether and methyl ethyl ether, with di-isopropyl ether being more preferred.

The anti-solvent is then combined with the solution, preferably by being added to the solution, to precipitate desloratadine. Preferred combinations of solvent/anti-solvent include chloroform/hexane or diisopropyl ether, or alternatively ethyl acetate/hexane. The ratio in the final product of the reaction might vary depending on the solvent/anti-solvent used, crystallization temperature and the temperature of the solution. When hexane is added to a solution of chloroform having a temperature above about 40° C., followed by crystallization at a temperature of from about 20° C. to about 30° C., the product contains from about 35% to about 40% Form II, in comparison to Form I. Preferably, the temperature of the solution is above about 40° C. for chloroform, more preferably from about 40° C. to about reflux (69° C.), and most preferably about 45° C. to about 55° C. When hexane or diisopropyl ether is added to chloroform having a temperature of below about 40° C., more preferably from about 20° C. to about 30° C., followed by cooling to a temperature of from about 0° C. to about 10° C., the product contains from about 2% to about 6% Form II. If ethyl acetate is used as a solvent, followed by addition of cold hexane and crystallization at a temperature below about 0° C., the resulting product has From II in the range of from about 15% to about 25%.

The resulting precipitate may then be recovered by techniques well known in the art, such as filtration, and optionally dried.

Form I having about a 2% to about 10%, more preferably about 4% Form II, may also be obtained by precipitation out of a C₁ to C₄ preferably ethanol through use of water as an anti-solvent. Water is preferably combined with ethanol at a temperature of from about 20° C. to about 30° C., followed by cooling to a temperature of from about 0° C. to about 10° C. The resulting precipitate may be recovered by techniques known in the art, and may optionally be dried.

Form I in a mixture with about 15% to about 25% of Form II may also be prepared by crystallization out of i-butyl acetate. A solution of desloratadine is prepared in i-butyl acetate, followed by crystallization. Preferably the solution is heated to reflux, followed by cooling to a temperature of from about 20° C. to about 30° C. The resulting crystals may then be recovered by techniques well known in the art, such as filtration, centifugation, decanting. The recovered crystals may also be dried.

A mixture of the two forms or Form I with less than about 10% Form II may also be prepared by crystallization out of isopropanol or i-butanol. A solution of desloratadine is prepared in isopropanol or i-butanol, followed by crystallization. Preferably, a solution is obtained by heating, followed by cooling to a temperature of about −10° C. to about 30°, more preferably from about 0° C. to about 25° C. When isopropanol is a solvent, cooling to a temperature of from 20° C. to about 30° C. results in about 5-6% Form II, while cooling to a temperature of from about −10° C. to about 10° C. results in about 2% of Form II. However, seeding with Form II increases the ratio of Form II to Form I. With isobutanol, cooling to a temperature of from about 20° C. to about 30° C. results in about 3% Form II. Thus, the present invention provides a process for increasing the amount of Form I by decreasing crystallization temperature. The resulting crystals may then be recovered by techniques well known in the art, such as filtration, centrifugation and decanting. The recovered crystals may also be dried. The drying results in a mixture that is approximately a 50:50 mixture.

When crystals obtained from crystallization from isopropanol and isobutanol are dried by storage at room temperature, a transition to a 1:1 mixture of Form I to Form II occurrs, despite the starting product having a much higher ratio of Form I to Form II. This transition suggests that a 50:50 mixture may be prepared by storing crystals obtained from at least C₁ to C₄ alcohols. Preferably, storage is carried out at a temperature of from about 20° C. to about 30° C., under reduced pressure. As used herein, the term “reduced pressure” refers to a pressure below about 100 mm Hg, more preferably of about 10 mm Hg to about 50 mm Hg.

The present invention also provides a process for preparing a mixture of desloratadine forms I and II by grinding desloratadine Form I. The duration of the grinding may vary depending on the desired final product and how the grinding is carried out. One of skill in the art may take an XRD after grinding to determine the optimal amount of time for grinding. Preferably, the grinding is carried out from about ½ an hour to about 3 hours. Usually, about 1 hour is sufficient to cause a transformation of Form I to Form II, i.e., more than about a 30% transformation of Form I by weight into Form II. In Example 14, the grinding results in about a 60% to about 70% mixture of Form II compared to Form I, more specifically about a 2:1 mixture. Substantially no polymorphic transformation occurred when grinding a mixture for up to about one minute. The grinding may be done by methods known in the art, such as manually in a mortar with pestle, or with any pressure generating device, such as a press.

Desloratadine may also be prepared in Form I or Form II, substantially free of the other form. As used herein, “substantially free” refers to having at most traces of the other form, i.e., less than about 1% weight of one polymorph to the other, more preferably less than about 0.5%, and most preferably less than about 0.1%. For example, Form I substantially free of Form II may be prepared by crystallization out of a suitable solvent. Desloratadine may be dissolved in an organic solvent such as acetonitrile, dimethylformamide, tetrahydrofuran and diethylcarbonate. The solvent may be heated to obtain a clear solution, preferably to reflux temperature (DMF is generally heated below its reflux temperature due to its high boiling point). Form I may then be recovered after crystallization. Crystallization may be induced for example by cooling the solvent, preferably to a temperature of from about 20° C. to about 30° C. The resulting crystals may then be dried under reduced or ambient pressure, preferably with slight heating.

Desloratadine Form II may also be prepared substantially free of Form I. In one embodiment, desloratadine is melted at a temperature above its melting temperature. The resulting molten material may then be allowed to cool naturally, such as at room temperature, or with a cooling apparatus to accelerate the cooling process. After cooling, preferably to a temperature of from about 20° C. to about 30° C., the molten material crystallizes into Form II substantially free of Form I. The resulting solidified material is preferably grounded.

Form II substantially free of Form I may be prepared by crystallization out of dimethylcarbonate. A solution of desloratadine in dimethylcarbonate is prepared, preferably by heating the dimethylcarbonate to about reflux temperature. The solution is then preferably cooled to a temperature of from about 20° C. to about 30° C. The resulting crystals may then be separated by techniques well known in the art, and optionally dried.

In another embodiment, a mixture of the two forms, i.e, from about a 35% to about 65% of both of the, may be prepared through combining a solution with a seeded antisolvent. In this embodiment, an anti-solvent is seeded with a mixture of desloratadine Form I and II, preferably a substantially equal mixture of the forms, and combined with a solution of desloratadine in a suitable solvent. A suitable solvent is isobutyl acetate. A suitable anti-solvent is a C₅ to C₁₂ aromatic or saturated hydrocarbon such as toluene or heptane.

The seeding allows for manipulating the ratio of Form I and II relative to each other. In example 19, the I-butyl acetate/heptane procedure without seeding produced 63% Form I, while the same procedure with seeding produced 37% and 42% Form I in examples 17 and 18 respectively. The seeding also allows for crystallization of a consistent ratio in that examples examples 17 and 18 are well within the 10% margin.

A mixture of two polymorphic forms, i.e., from about 25 to about 75% of form of both forms, may be obtained by crystallization from a mixture of a solvent of desloratadine (e.g. toluene, ethyl acetate, iso-butyl acetate) and a co-solvent (a C₁ to C₄ alcohol such as iso-propanol and methanol). The polymorphic form ratio of the final product may be influenced by amount of the co-solvent. Preferably, the amount of the co-solvent is from about 3% to about 13% by volume, more preferably from about 7% to about 10% by volume. In example 21 22, 23 and 24 different solvents are used to obtain a mixture of polymorphic forms. In example 21, 10% iso-propanol-iso-butyl acetate is used. In example 22, 10% iso-propanol-toluene is used. In example 23, 5% methanol-toluene is used. In example 24, 3% iso-propanol is used.

The use of a co-solvent, particularly a C₁ to C₄ alcohol, allows for manipulating ratio of Form II compared to Form I. Crystallization out of i-butyl acetate for example results in about 15-25% of Form II, while with addition of 10% isopropanol results in about 40-50% Form II. Crystallization out of ethyl acetate results almost entirely in Form II, while use of 3% isopropanol as a co-solvent allows for obtaining about 70-80% of Form I.

The present invention provides a process for the preparation of a mixture of Form I and Form II. Preferably, the mixture is of about 35-82% desloratadine Form I and about 18-65% desloratadine Form II. More preferably, the mixture is of about 55-82% desloratadine Form I and 18-45% desloratadine Form II.

When using this process for industrial purposes, it may be advantageous to start with a desloratadine salt as a starting material since the salt is easy to handle. The desloratadine free base may then be obtained from the salt, such as the acetate salt.

Desloratadine free base may be obtained by combining a desloratadine salt, such as desloratadine acetate, with an organic solvent selected from a C₂ to C₅ alkyl acetate, butanol, isobutanol, toluene and chloroform, followed by addition of an aquoues base at a temperature of about 50° C. to about 70°, preferably about 60° C. to form two phases. Preferably, the organic solvent is selected from the group consisting of isobutyl acetate, ethyl acetate, butanol, isobutanol, toluene and chloroform. Most preferably, the organic solvent is isobutyl acetate. Suitable bases include those of alkali metal and alkaline earth metals such as potassium, sodium and calcium oxide/hydroxide/carbonate, preferably sodium or potassium hydroxide. Preferably, a solution of about 2% to about 6% of sodium or potassium hydroxide, preferably about a 4% solution may be used. After addition of the base, the organic phase containing desloratadine is then recovered.

The organic phase may be purified by filtration. The filtrate is then preferably concentrated, more preferably by evaporation under reduced pressure. An additional amount of the organic solvent described above (the exact amount is calculated from the determined actual concentration) may be added to the solution or some of the solvent is evaporated in order to reach an optimum concentration. The concentration is preferably of about 5 m/m % to about 40 m/m %, more preferably about 15 m/m %.

In one embodiment, the process for preparing said mixture of Form I and Form II comprises providing, at an elevated temperature, a solution of desloratadine free base in an organic solvent selected from a C₂ to C₅ alkyl acetate, butanol, isobutanol, toluene and chloroform. The solution is then combined with a C₅ to C₁₂ cyclic or acylcic saturated hydrocarbon such as heptane or hexane. The difference in temperature is preferably at least about 40° C. The temperature of the solution is preferably of about 60° C. to about 100° C. and most preferably about 90° C. The temperature of the hydrocarbon is preferably about −10° C. to about 20° C., more preferably about 0° C. The difference between the temperature of the solvent and anti-solvent maintains the percentage of each form within the desired range, so that an equilibrium between the 2 forms is not obtained. The addition is also preferably carried out in less than about 10 minutes to even better retain the desired range.

Desloratadine then precipitates from the solution. The resulting heterogeneous mixture may then be stirred and the desloratadine recovered by conventional techniques in the art, such as filtration.

The desloratadine obtained may then be dried. Drying may be carried out under ambient or reduced pressure. Further, desloratadine may be heated or put in a steam of air to accelarate the drying process. Heating the wet desloratadine obtained stabilizes the final product. Heating is preferably carried out at a temperature of about 30° C. to about 50° C., more preferably about 40° C.

The processes of the present invention yields a product with a low level of residual solvent. The amount of solvents in the product is preferably less than about 5000 ppm of any solvent, more preferably about 50 ppm to about 4000 ppm. Examples of such solvents include isobutyl acetate, n-heptane, n-hexane, ethyl acetate, butanol, isobutanol, toluene, chloroform and combinations thereof. Preferably, the desloratadine contains a low level of isobutyl acetate and/or heptane, most preferably the desloratadine contains a low level of isobutyl acetate, which is the solvent in the above process. Such low levels of residual solvent contribute to the stability of a mixture of Form I and Form II.

In one embodiment, these mixtures substantially free of solvents are obtained on an industrial scale. Industrial sized batch of the present invention is preferably at least about 0.5 Kg, more preferably at least about 1 Kg and most preferably at least about 10 Kg.

The present invention provides a mixture of about 35-82% desloratadine Form I and about 18-65% desloratadine Form II, containing about 50 ppm to about 4000 ppm of any one of isobutyl acetate, n-heptane, n-hexane, ethyl acetate, butanol, isobutanol, toluene, chloroform and combinations thereof. Preferably, the mixture is of about 55-82% desloratadine Form I and about 18-45% desloratadine Form II.

One skilled in the art may also appreciate that the present invention is not limited by the order of the additions in adding an anti-solvent. For example, a solution may be added to an anti-solvent or vice versa, though an embodiment may prefer one over the other. Crystallization of a compound is often better when a solution is added to the anti-solvent, but operationally it is often more convenient to add the anti-solvent to the solution. When adding an anti-solvent to a residue, the order of addition is of minimal relevance. The term “combining” encompasses both orders of addition.

Many processes of the present invention involve crystallization out of a particular solvent, i.e., obtaining a solid from a solution. Crystallization may occur spontaneosuly or be induced. The present invention covers both embodiments where crystallization or precipitation occurs spontaneously, or is induced/accelerated, unless if such inducement is critical for obtaining a particular polymorph.

The starting material used for the processes of the present invention may be any crystalline or other form of desloratadine, including various solvates and hydrates. With crystallization processes, the crystalline form of the starting material does not usually affect the final result since the original crystalline form is lost once a material goes into solution. With a slurry/trituration process, the starting material sometimes makes a difference, since without complete dissolution, the original crystal form may not be completely lost.

Pharmaceutical compositions of the present invention contain desloratadine Form I and/or Form II. The desloratadine prepared by the processes of the present invention are ideal for pharmaceutical composition. In addition to the active ingredient(s), the pharmaceutical compositions of the present invention may contain one or more excipients.

Excipients are added to the composition for a variety of purposes. Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminium silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, desloratadine and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

Capsules, tablets and lozenges, and other unit dosage forms preferably contain from about 2 to about 20 mg of desloratadine, more preferably about 2 mg to about 10 mg of desloratadine, and most preferably about 5 mg.

### EXAMPLES

Instrumentation

X-Ray powder diffraction data were obtained using a SCINTAG powder X-Ray diffractometer model X'TRA equipped with a solid state detector. Copper radiation of 1.5418 Å was used. A round aluminum sample holder with round zero background quartz plate, with cavity of 25(diameter)*0.5(dept) mm.

DSC analysis was done using a Mettler 821 Star^{e}. The weight of the samples was about 5 mg; the samples were scanned at a rate of 10° C./min from 30° C. to 250° C. The oven was constantly purged with nitrogen gas at a flow rate of 40 ml/min. Standard 40 μl aluminum crucibles covered by lids with 3 holes were used.

IR analysis was done using a Perkin Elmer SPECTRUM ONE FT-IR spectrometer in DRIFTt mode. The samples in the 4000-400 cm⁻¹ interval were scanned 64 times with 4.0 cm⁻¹ resolution

In the following examples, the vacuum oven used had a pressure of approximately 30 mm Hg and the refrigerator had a temperature of about 5° C.

Preparation of Desloratadine Form I

### Example 1

Preparation of Desloratadine Form I—Dry

A solution of desloratadine (5.0 grams) in acetonitrile (54 ml) was heated at reflux temperature until complete dissolution. Charcoal (0.52 grams) was added to the clear solution, heated for a few minutes, and filtered. After filtration, the solution was heated again to reflux temperature, and allowed to cool to room temperature, resulting in crystallization. The resulting precipitate was filtered, and washed with 10 ml of ethyl acetate-n-hexane (3:1). The wet solid product was dried in a vacuum oven at room temperature. The X-Ray Powder diffraction showed that the sample had crystallized in polymorphic Form I (4.01 g). [The solvent to solute ratio is preferably from about 8 to about 14 ml/g]

### Example 2

Preparation of Desloratadine Form I—Dry

A solution of desloratadine (5.0 grams) in dimethylformamide (19 ml) was heated at 75° C. until complete dissolution. Charcoal (0.55 grams) was added to the solution, heated for a few minutes, and filtered. After filtration, the solution was heated again, and allowed to cool to room temperature, allowing for crystallization. The resulting precipitate was filtered, and washed with 10 ml of ethyl acetate: n-hexane (1:1). The wet solid product was then dried in a vacuum oven at 40° C. The X-Ray Powder Diffraction pattern showed that the sample was crystallized in polymorphic Form 1 (3.33 g). [The solvent to solute ratio is preferably from about 3 to about 10 ml/g]

### Example 3

Preparation of Desloratadine Form I—Wet and Dry

A solution of desloratadine (3.0 grams) in tetrahydrofuran (7.5 ml) was heated at reflux temperature (66° C.) until complete dissolution. The clear solution was allowed to cool to room temperature, resulting in crystallization. The resulting precipitate was filtered and was analyzed by X-Ray Powder Diffraction, and found to be Form I. The wet solid product was dried in a vacuum oven at room temperature. The X-Ray Powder diffraction showed that the sample had crystallized in polymorphic Form I (1.73 g). [The solvent to solute ratio is preferably from about 1 to about 5 ml/g]

### Example 4

Preparation of Desloratadine Form I—Wet and Dry

A solution of desloratadine (3.0 grams) in diethyl carbonate (15 ml) was heated at reflux temperature (126° C.) until complete dissolution. The clear solution was allowed to cool to room temperature, resulting in crystallization. The resulting precipitate was filtered and was analyzed by X-Ray Powder Diffraction, and found to be Form I. The wet solid product was dried in a vacuum oven at room temperature. The X-Ray Powder diffraction showed that the sample had crystallized in polymorphic Form 1 (2.32 g). [The solvent to solute ratio is preferably from about 2 to about 8 ml/g]

Preparation of a Mixture of Desloratadine Form I and Form II in which Form II was about 2-10%

### Example 5

Preparation of Desloratadine Form I—Wet and Dry

Desloratadine (3.0 grams) in chloroform (15 ml) was dissolved at room temperature, resulting in a clear solution. n-hexane was added to the solution until complete precipitation. The resulting mixture was kept in the refrigerator overnight. The resulting precipitate was filtered and was analyzed by X-Ray Powder Diffraction, and found to be Form I. A trace of Form II was present in the sample, as evident by XRPD pattern. The wet solid product was then dried in a vacuum oven at room temperature for about 15 hours. The X-Ray Powder Diffraction Pattern showed that the sample had crystallized in polymorphic Form I (1.85 g). A trace of Form II was present in the sample as evident by XRPD (approximately 2% wt/wt Compared to Form I). [The solvent (chloroform) to solute ratio is preferably from about 2 to about 8 ml/g].

### Example 6

Preparation of Desloratadine Form I—Wet and Dry

Desloratadine (3.0 grams) in chloroform (15 ml) was dissolved at room temperature to obtain a clear solution. Di-isopropyl ether was added to the solution until complete precipitation. The resulting mixture was kept in the refrigerator. The resulting precipitate was filtered and analyzed by X-Ray Powder Diffraction and found to be Form I. A trace amount of Form II was present in the sample as evident by the XRPD pattern. The wet solid product was then dried in a vacuum oven at room temperature for about 15 hours. The X-Ray Powder Diffraction pattern showed that the sample had crystallized in polymorphic Form I. (1.35 g). A trace amount of Form II was also present in the sample as evident by XRPD. (approximately 6% wt/wt Compared to Form I). [The solvent (chloroform) to solute ratio is preferably from about 2 to about 8 ml/g].

### Example 7

Preparation of Desloratadine Form I—Wet and Dry

A solution of desloratadine (3.0 grams) in absolute ethanol (13 ml) was stirred at room temperature until complete dissolution. Water was added to the clear solution until complete precipitation, and the resulting mixture was kept in the refrigerator overnight. The resulting precipitate was filtered and analyzed by X-Ray Powder Diffraction and found to be Form I. A trace of Form II was present in the sample as evident by XRPD pattern. The wet solid product was then dried in a vacuum oven at room temperature for about 15 hours. The X-Ray Powder Diffraction pattern showed that the sample was crystallized in polymorphic Form I. (1.64 g). A trace of Form II was present in the sample as evident by XRPD. (approximately 4% wt/wt Compared to Form I). [The solvent (ethanol) to solute ratio is preferably from about 2 to about 6 ml/g].

Preparation of a Mixture of Desloratadine Form I and Form II in the Ratio of Approximately 3-6 to 1, i.e., about 15-25% of Form II in the Form I

### Example 8

Preparation of a Mixture of Desloratadine Form I and II

A solution of desloratadine (5.0 grams) in ethyl acetate (25 ml) was heated at reflux temperature until complete dissolution. The warm solution was added to 50 ml of cold n-hexane (−13° C.) resulting in precipitation. The mixture was kept at −13° C. for 1 hour and filtered. The wet solid product was dried in a vacuum oven at room temperature overnight. The X-Ray Powder Diffraction Pattern showed that the sample had crystallized as a mixture of polymorphic Form I and Form II in the ratio of approximately 4 to 1. [The solvent (ethyl acetate) to solute ratio is preferably from about 3 to about 10. The ratio of ethyl acetate to hexane is preferably from about 1 to about 5 ml/g].

### Example 9

Preparation of a Mixture of Desloratadine Form I and II

A solution of desloratadine (3.0 grams) in isobutyl acetate (15 ml) was heated at reflux temperature (120° C.) until complete dissolution. The clear solution was allowed to cool to room temperature, resulting in crystallization. The resulting precipitate was filtered and was analyzed by X-Ray Powder Diffraction, and found to be mixture of Form 1 and Form 2. The wet solid product was dried in a vacuum oven at room temperature. The X-Ray Powder diffraction showed that the sample had crystallized in polymorphic Form I and Form 2 in the ratio of approximately 5.5 to 1 (2.13 g). [The solvent to solute ratio is preferably from about 3 to about 8 ml/g].

Preparation of a Mixture of Desloratadine Form I and Form II in the Ratio of Approximately 1 to 1

### Example 10

Solution of desloratadine (3.0 grams) in isopropanol (6 ml) was heated at reflux temperature (80° C.) until complete dissolution. The solution was allowed to cool to room temperature, resulting in crystallization of a solid material. The solid material was filtered and analyzed by X-Ray powder diffraction and found to be a mixture of Form 1 and Form 2 (Form 2 was 5-6% in the Form 1). The wet solid product was dried in a vacuum oven at room temperature overnight. The X-Ray Powder diffraction pattern showed that the sample was crystallized in mixture of polymorphic Form 1 and Form 2 in the ratio of approximately 1 to 1 (1.5 grams).

### Example 11

Solution of desloratadine (10.0 grams) in isopropanol (25 ml) was heated at reflux temperature (80° C.) in a glass reactor until complete dissolution. The warm solution was cooled to 0° C. during 30 minutes resulting in crystallization of a solid material. The solid material was filtered and analyzed by X-Ray powder diffraction and found to be a mixture of Form 1 and Form 2 (Form 2 was 2% in the Form 1). The wet solid product was dried in a vacuum oven at room temperature overnight. The X-Ray Powder diffraction pattern showed that the sample was crystallized in mixture of polymorphic Form 1 and Form 2 in the ratio of approximately 1 to 1 (8.7 grams).

### Example 12

Solution of desloratadine (3.0 grams) in 2-methyl-1 propanol (isobutanol) (3 ml) was heated at reflux temperature (110° C.) until complete dissolution. The warm solution was allowed to cool to room temperature resulting in crystallization of a solid material. The solid material was filtered and analyzed by X-Ray powder diffraction and found to be a mixture of Form 1 and Form 2 (Form 2 was 3% in the Form 1). The wet solid product was dried in a vacuum oven at room temperature overnight. The X-Ray Powder diffraction pattern showed that the sample was crystallized in mixture of polymorphic Form 1 and Form 2 in the ratio of 1 to 1 (2.7 grams).

### Example 13

Preparation of a Mixture of Desloratadine Form I and II

Desloratadine (3.0 grams) in chloroform (5 ml) was heated at 50° C. until complete dissolution. n-hexane was added to the warm clear solution until complete precipitation. The resulting mixture was allowed to cool to room temperature, and kept in the refrigerator overnight and filtered. The filtered material was analyzed by X-Ray Powder Diffraction, which showed a mixture of Form I and Form II. (Form II was 38% in the Form 1). The wet solid product was dried in a vacuum oven at room temperature for about 15 hours. The X-Ray Powder Diffraction pattern showed that the sample had crystallized as a mixture of polymorphic Form I and Form II in the ratio of 1.5 to 1 (2.32 g). [The solvent to solute ratio is preferably from about 1 to about 5 ml/g].

### Example 14

Preparation of a Mixture of Desloratadine Form I and II

Desloratadine Form I (1.5 grams) was ground in a mortar. After 1 hour, a sample was taken, and the X-Ray Powder diffraction pattern of the sample showed that the sample had crystallized as a mixture of polymorphic Form II and Form I (Form II was approximately 65 wt/wt Compared to Form I). Another sample taken after 1.5 hours showed, according to X-Ray Powder Diffraction, that the sample had crystallized as a mixture of polymorphic Form II and Form I. (Form II was approximately 63 wt/wt Compared to Form I).

Preparation of Desloratadine Form II

### Example 15

Preparation of Form II

Desloratadine (5.0 grams), in the solid state, was heated at a temperature of 160° C. until all of the solid material had melted. The resulting clear liquid was allowed to cool slowly to room temperature, which solidified. The resulting solid material was ground in a mortar. The X-Ray Powder Diffraction pattern showed that the sample had crystallized in polymorphic Form II.

### Example 16

### Wet and Dried

Preparation of Desloratadine Form II—Wet and Dry

A solution of desloratadine (3.0 grams) in dimethyl carbonate (26 ml) was heated at reflux temperature (90° C.) until complete dissolution. The clear solution was allowed to cool to room temperature, resulting in crystallization. The resulting precipitate was filtered and was analyzed by X-Ray Powder Diffraction, and found to be Form II. The wet solid product was dried in a vacuum oven at room temperature. The X-Ray Powder diffraction showed that the sample had crystallized in polymorphic Form II (2.14 g).

### Example 17

Preparation of Desloratadine Form I and Form II

A solution of desloratadine (20 grams) in iso-butyl acetate (120 ml) was heated in the glass reactor at 100° C. until complete dissolution. In another glass reactor the mixture of form I and form II of desloratadine (2 g) was suspended in n-heptane (80 ml) at 35° C. The hot iso-butyl acetate solution was dropped into n-heptane slurry (temperature of slurry was between 30-40° C.). The resulting crystalline product was filtered and was dried in a vacuum oven at room temperature. The X-Ray Powder Diffraction showed that the sample had crystallized in as a mixture of polymorphic Form I and Form II (16 g). Mixture of Form I and Form II was in the ratio of 37 to 63. [The solvent (isobutyl acetate) to solute ratio is preferably from about 6 to about 1 ml/g. The ratio of iso-butyl acetate to n-heptane is preferably from about 3:2 (v/v)]

### Example 18

Preparation of Desloratadine Form I and Form II

A solution of desloratadine (400 grams) in iso-butyl acetate (2400 ml) was heated in the glass reactor at 100° C. until complete dissolution. In another glass reactor the mixture of form I and form II of desloratadine (40 g) was suspended in n-heptane (1600 ml) at 35° C. The hot iso-butyl acetate solution was dropped into n-heptane slurry (temperature of slurry was between 30-40° C.). The resulting crystalline product was filtered and was dried in a vacuum oven at room temperature for about 15 hours. The X-Ray Powder diffraction showed that the sample had crystallized in as a mixture of polymorphic Form I and Form II (353 g). Form I and Form II was in the ratio of 42 to 58. [The solvent (iso-butyl acetate) to solute ratio is preferably from about 6 to about 1 ml/g. The ratio of iso-butyl acetate to n-heptane is preferably from about 3:2 v/v]

### Example 19

Preparation of Desloratadine Form I and Form II

A solution of desloratadine (10.0 grams) in isobutyl acetate (60 ml) was heated at 100° C. until complete dissolution. The hot isobutyl acetate solution was dropped into n-heptane at 35° C. (temperature of n-heptane was between 30-40° C.). The resulting crystalline product was filtered and was dried in a vacuum oven at room temperature. The X-Ray Powder Diffraction pattern showed that the sample was crystallized in as a mixture of polymorphic Form I and Form II (6.0 g) in the ratio of 63 to 37. [The solvent (isobutyl acetate) to solute ratio is preferably from about 6 to about 1 ml/g. The ratio of isobutyl acetate to n-heptane is preferably from about 3:2]

### Example 20

Preparation of Desloratadine Form I and Form II

A solution of desloratadine (10.0 grams) in iso-butyl acetate (60 ml) was heated at 100° C. until complete dissolution. In another glass reactor the mixture of form I and form II of desloratadine (10 g) was suspended in iso-butyl acetate (60 ml) at 40° C. The hot iso-butyl acetate solution was dropped into iso-butyl acetate slurry (temperature of slurry was between 30-50° C.). The resulting crystalline material was filtered and was dried in a vacuum oven at room temperature. The X-Ray Powder diffraction showed that the sample had crystallized in as a mixture of polymorphic Form I and Form II (13.7 g). Form I and Form II was in the ratio of 59 to 41. [The solvent to solute ratio is preferably from about 1 to about 6 ml/g].

### Example 21

Preparation of Desloratadine Form I and Form II

A solution of desloratadine (10.0 grams) in 10% iso-propanol-iso-butyl acetate (50 ml) was heated at 100° C. until complete dissolution. The clear solution was allowed to cool to room temperature, resulting in crystallization. The resulting precipitate was filtered and was dried in a vacuum oven at room temperature. The X-Ray Powder diffraction showed that the sample had crystallized in as a mixture of polymorphic Form I and Form II. (6.1 g) in the ratio of 56 to 44. [The solvent to solute ratio is preferably from about 5 to about 1 ml/g].

### Example 22

Preparation of Desloratadine Form I and Form II

Desloratadine (20.0 grams) in 10%-iso-propanol-toluene (50 ml) was dissolved at 80° C. until complete dissolution. The clean solution was allowed to cool to room temperature, resulting in crystallization. The crystalline material was filtered and was dried in a vacuum oven at room temperature for about 15 hours. The X-Ray Powder Diffraction Pattern showed that the sample had crystallized in as a mixture of polymorphic Form I and Form II (8.9 g) in the ratio of 50 to 50. [The solvent (10% iso-propanol-toluene) to solute ratio is preferably from about 2.5 to about 1 ml/g].

### Example 23

Preparation of Desloratadine Form I and Form II

Desloratadine (10.0 grams) in 5% methanol-toluene (20 ml) was dissolved at 100° C. to obtain a clear solution. The resulting solution was allowed to cool to 20° C. for 30 minutes. The resulting crystalline material was filtered and dried in a vacuum oven at room temperature for about 16 hours. The X-Ray Powder Diffraction pattern showed that the sample had crystallized in as a mixture of polymorphic Form I and Form II (5.7 g) in the ratio of 53 to 47. [The solvent (methanol-toluene) to solute ratio is preferably from about 2 to about 1 ml/g.]

### Example 24

Preparation of Desloratadine Form I and Form II

Desloratadine (32.5 grams) in mixture of iso-propanol (6 ml) and ethyl acetate (200 ml) was dissolved at 80° C. to obtain a clear solution. Charcoal (1.0 gram) was added to the clear solution, heated for a few minutes, and filtered. After filtration, the crystallization started immediately from solution. The resulting crystalline material was filtered and was dried in a vacuum oven at room temperature for about 16 hours. The X-Ray Powder Diffraction pattern showed that the sample had crystallized in as a mixture of polymorphic Form I and Form 11 (23.3 g) in the ratio of 74 to 26.

### Example 25

Preparation of Desloratadine as a Mixture of Polymorphs from N-methyl Desloratadine [with Loratadine as an Intermediate]

N-methyl desloratadine (40 g) was dissolved in 200 ml anhydrous toluene at 60° C. A mixture of 30 ml ethyl chloroformate and 60 ml anhydrous toluene was added dropwise for 60 minutes. After the addition had been completed, the reaction was checked for conversion. Because of the incomplete conversion, a mixture of 2.5 ml ethyl chloroformate and 5 ml anhydrous toluene was added dropwise for 10 minutes.

The reaction mixture was filtered off and the filtrate was concentrated at reduced pressure. Repeated co-distillation with 2-propanol resulted in the removal of toluene. The residue was dissolved in 150 ml 2-propanol and heated to reflux. In the meantime, 16 g KOH was added. When the reaction mixture reached the reflux temperature, another 16 g KOH was added. After 1 h another 8 g KOH was added and the mixture was heated for 1 h. The reaction was checked for conversion, and since the conversion was incomplete, 8 g KOH was added to the mixture and it was refluxed for an additional hour. The reaction mixture was cooled to 60° C. and 60 ml water was added. After the solid had been dissolved, the two phases of the liquid were separated. The lower (aqueous) phase was extracted twice with 100 ml ethylacetate. These ethylacetate extracts were combined and used to dissolve the residue that was obtained by the concentration of the original upper (2-propanol) phase to about half of its original weight. This was washed successively with 150 ml water, 100 ml water and 100 ml brine prior to drying over 20 g K₂CO₃. The drying agent was filtered off and the filtrate was seeded with a mixture of Form I and II, and combined slowly with 350 ml n-heptane with stirring. Crystallization started slowly, after 1-2 hours the suspension was taken to refrigerator. The resulting crystalline product was filtered off and dried in a vacuum oven at room temperature. Yield: 14.20 g (40%). The X-ray Powder Diffraction measurement showed that the ratio of the two polymorphic forms was 62:38 (Form 1/Form 2).

### Example 26

Preparation of Desloratadine as a Mixture of Polymorphs from Loratadine

Loratadine (40 g) and KOH (16 g) were suspended in 150 ml 2-propanol and heated to reflux. Half an hour later than the reaction mixture reached the reflux temperature, another 16 g KOH was added. After 1 hour, another 8 g KOH was added and the mixture was heated for 1 hour. The reaction was checked for conversion. The conversion was complete. The reaction mixture was cooled to 60° C., and 60 ml water was added. After the solid had dissolved, the two phases of the liquid were separated. The lower (aqueous) phase was extracted twice with 100 ml ethylacetate. These ethylacetate extracts were combined and used to dissolve the residue that was obtained by the concentration of the original upper (2-propanol) phase to about half of its original weight. This was washed successively with 150 ml water, 100 ml water and 100 ml brine. The combined aqueous washings were extracted with 50 ml ethylacetate which was added to the other ethylacetate extract. This was dried over 20 g K₂CO₃, decanted and dried again over 20 g K₂CO₃. The drying agent was filtered off and the filtrate was seeded and after 15 minutes it was combined slowly with 150 ml n-heptane with stirring overnight at room temperature. The resulting crystalline product was filtered off and dried in a vacuum oven at room temperature. Yield: 12.85 g (40%). The X-ray Powder Diffraction measurement showed that the ratio of the two polymorphic forms was 77:23 (Form 1/Form 2).

### Example 27

Preparation of Desloratadine as a Mixture of Polymorphs from Loratadine

Loratadine (40 g) and KOH (16 g) was suspended in 150 ml 2-propanol and heated to reflux. Half an hour after reaching reflux temperature, another 16 g KOH was added. After 1 h, another 8 g KOH was added and the mixture was heated for another 1 hour. The reaction was checked for conversion. The conversion was complete.

The reaction mixture was cooled to 60° C. and 60 ml water was added. After the solid had been dissolved, the two phases of the liquid were separated. The lower (aqueous) phase was extracted twice with 100 ml ethylacetate. These ethylacetate extracts were combined and used to dissolve the residue that was obtained by the concentration of the original upper (2-propanol) phase to about half of its original weight. This was washed successively with 150 ml water, 100 ml water and 100 ml brine. The combined aqueous washings were extracted with 50 ml ethylacetate which was added to the other ethylacetate extract. This was dried over 20 g K₂CO₃, decanted and dried again over 20 g K₂CO₃. The drying agent was filtered off and the filtrate was concentrated at 50° C. under vacuum to obtain a relatively concentrated solution (estimated concentration: 25 m/m %). This was seeded with a mixture of Form I and Form II, and after 15 minutes it was combined slowly with 60 ml n-heptane with stirring overnight at room temperature. The resulting crystalline product was filtered off and dried in a vacuum oven at room temperature. Yield: 21.13 g (65%). The X-ray Powder Diffraction measurement showed that the ratio of the two polymorphic forms is 43:57 (Form 1/Form 2).

### Example 28

Preparation of Desloratadine as a Mixture of Polymorphs from Loratadine

To 2-propanolic (750 ml) solution of loratadine (200 g), KOH (80 g) was added while stirring, and heated to reflux (jacket: 95° C.) temperature. After 60 minutes, KOH (80 g), and after 120 minutes, KOH (40 g), were added to the reaction mixture. When the reaction was complete, the mixture was cooled to 50° C. and diluted with distilled water (300 ml). The two-phase mixture was stirred until complete dissolution of the solids. The lower (aqueous) phase was removed and the 2-propanolic phase was evaporated at vacuum (jacket: 50° C.). The resulting material was diluted with toluene (800 ml) and washed successively with distilled water (2×300 ml) at 50° C. From the resulting solution, 200 ml of solvent was evaporated at vacuum (jacket: 50° C.) to 150 ml residual volume. The residual volume was heated to 80° C., and 2-propanol (4.5 ml) was added, and cooled to 20° C. for 1 hour. After the combining and cooling, the crystalline material was filtered off and dried in vacuum at 25° C. The X-Ray Powder Diffraction showed that the sample had crystallized as a mixture of polymorphic Form I and Form II (15.6 g). Mixture of Form I and Form II was in the ratio of 39 to 61.

### Example 29

Preparation of Desloratadine as a Mixture of Polymorphs from Loratadine

To 2-propanolic (750 ml) solution of loratadine (200 g) KOH (80 g) was added during stirring and heated to reflux (jacket: 95° C.). After 60 minutes KOH (80 g), 120 minutes KOH (40 g) were added to the reaction mixture. When the reaction was complete (after 5 hours), mixture was cooled to 50° C. and diluted with distilled water (300 ml). Two-phase mixture was stirred until complete dissolution of the solids. The lower (aqueous) phase was removed and the 2-propanolic phase was evaporated at vacuum (jacket: 50° C.). The resulting material was diluted with toluene (800 ml) and washed successively with distilled water (2×300 ml) at 50° C. From the resulting solution 150 ml solution was evaporated at vacuum (jacket: 50° C.) to 95 ml residual volume, which was then heated to 90° C. 2-propanol (10 ml) was added and cooled to 20° C. and kept at this temperature for 30 minutes. A crystalline material was filtered off and dried in vacuum at 25° C. The X-Ray Powder Diffraction showed that the sample had crystallized in as a mixture of polymorphic Form I and Form 11 (9.8 g). Mixture of Form I and Form II was in the ratio of 25 to 75.

### Example 30

Preparation of Desloratadine Mixture Form I and Form II

A slurry of desloratadine acetate (20 grams) in toluene (100 ml) and 3.8% KOH solution (95,6 ml) was heated and stirred in the glass reactor at 60° C. until complete dissolution. After phase separation, toluene solution of desloratadine was washed with distilled water (60 ml) at 60° C. The resulting toluene solution was concentrated by vacuum (jacket: 60° C.) to dryness. The solid material was dissolved in toluene-2-propanol 9:1 (74 ml) at 60° C. The warm solution was cooled to 20° C. for 4 hours and stirred at this temperature for 8 hours. This slurry was warmed again to 45° C. In another glass reactor n-heptane (100 ml) was cooled to 0° C. The warm slurry of desloratadine in toluene was dropped into cold n-heptane (temperature of slurry was between 0-12° C.), and it was stirred at 0° C. for 1 hours. The resulting crystalline product was filtered and dried in a vacuum oven at room temperature. The X-Ray Powder Diffraction showed that the sample had crystallized in as a mixture of polymorphic Form I and Form II (ratio 76-24). (13.2 g, 79%, HPLC purity: 99.9%).

### Example 31

Preparation of Desloratadine Mixture Form I and Form II

Desloratadine was prepared from desloratadine acetate according to the example 1. The X-Ray Powder Diffraction showed that the sample had crystallized in as a mixture of polymorphic Form I and Form II (10.8 g, 65%, HPLC purity: 99.8%). Mixture of Form I and Form II was in the ratio of 42 to 38.

### Example 32

Preparation of Desloratadine Mixture Form I and Form II

A slurry of desloratadine acetate (20 grams) in toluene (100 ml) and 3.8% KOH solution (75 ml) was heated and stirred in the glass reactor at 60° C. until complete dissolution. After phase separation, toluene solution of desloratadine was washed with distilled water (60 ml) at 60° C. The resulting toluene solution was concentrated by vacuum (jacket: 60° C.) to dryness. The solid material was dissolved in toluene-2-propanol 9:1 (50 ml) at 60° C. The warm solution was cooled to 20° C. for 4 hours and stirred at this temperature for 10 hours. This slurry was warmed again to 50° C. The resulting toluene slurry was concentrated by vacuum (jacket: 50° C.) to half of volume. In another glass reactor n-heptane (100 ml) was cooled to 0° C. The warm slurry of desloratadine in toluene was dropped into cold n-heptane (temperature of slurry was between 0-12° C.), and it was stirred at 0° C. for 1 hours. The resulting crystalline product was filtered and was dried in a vacuum oven at room temperature. The X-Ray Powder Diffraction showed that the sample had crystallized in as a mixture of polymorphic Form I and Form II (64 to 36%) (10.6 g, 63%) HPLC purity: 99.7%.

### Example 33

Preparation of Desloratadine Mixture Form I and Form II

Desloratadine acetate (400 g) was stirred in iso-butyl acetate (3000 ml) at 60° C. with 5% aqueous solution of NaOH (1000 ml, 1.2 eq). When the solid material was dissolved, the aqueous phase was removed and the organic layer was washed with distilled water (1200 ml). After phase separation the organic phase is filtered at 60° C. and the filtrate is concentrated in vacuum at 95±5° C.

A 10 ml sample of the solution is evaporated to dryness in order to check the concentration of the solution. More iBuOAc (the exact amount is calculated from the determined actual concentration) was added to the solution or some of the solvent was evaporated in order to reach 16.0 m/m %. The temperature of the solution was maintained at 95±5° C.

The hot solution was added to 10000 ml n-heptane (temperature: 0±5° C., stirring: 250-300 rpm) in a period of 2.0-5.5 min. The suspension was stirred for an additional ˜10 min then filtered. The wet solid was dried at 40° C. in vacuum, where can use a slight stream of air or nitrogen in order to continuously remove the vapors from the oven. Drying time: 10 h. Residual solvents: 2700 ppm iso-butyl acetate; 3750 ppm n-heptane.

Study of Stability of Desloratadine Polymorphs

The desired mixture of polymorphic forms (Form 1 and Form 2) of desloratadine was prepared by different methods in order to investigate a stability of polymorphic ratio of desloratadine. Stability of ratio of the mixture of desloratadine polymorphic forms at two different conditions:

1.) 25° C., 60% RH

2.) 40° C., 75% RH

In all cases samples were packed in polyethylene and store above conditions.

### Example 1 of Stability Tests

Recrystallization of desloratadine (3 g) from dimethyl carbonate-diethyl carbonate (1:1) (35 ml) at 110° C. obtained a mixture. The X-ray Powder Diffraction measurement showed that the ratio of the two polymorphic forms was 57:43 (Form 1/Form 2).

| | | 25° C. 60% RH | | | |
|---|---|---|---|---|---|
| | Time | Form | | 40° C. 75% RH | |
| Time | (weeks) | 1 (%) | Form 2 (%) | Form 1 (%) | Form 2 (%) |
| 0 week | 0 | 57 | 43 | 57 | 43 |
| 1 week | 1 | 59 | 41 | 46 | 54 |
| 2 weeks | 2 | 53 | 47 | 43 | 57 |
| 1 month | 4 | 55 | 45 | 50 | 50 |
| 2 months | 8 | 63 | 37 | 39 | 61 |
| 3 months | 12 | 52 | 48 | | |

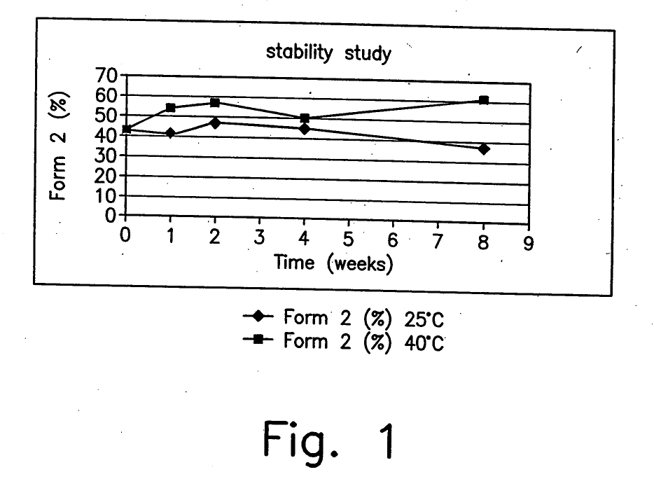

Stability study is disclosed in FIG. 1.

### Example 2 of Stability Tests

Recrystallization of desloratadine (3 g) from iso-propanol-n-heptane (1:1) (111 ml) at 85° C. obtained a mixture. The X-ray Powder Diffraction measurement showed that the ratio of the two polymorphic forms was 62:38 (Form 1/Form 2).

| | | 25° C., 60% RH | | 40° C., 75% RH | |
|---|---|---|---|---|---|
| | Time | Form 1 | Form 2 | Form 1 | Form 2 |
| Time | (weeks) | (%) | (%) | (%) | (%) |
| 0 week | 0 | 62 | 38 | 62 | 38 |
| 1 week | 1 | 65 | 35 | 63 | 37 |
| 2 weeks | 2 | 61 | 39 | 64 | 36 |
| 1 month | 4 | 66 | 34 | 65 | 35 |
| 2 months | 8 | 67 | 33 | 65 | 35 |
| 3 months | 12 | 66 | 34 | | |

### Example 3 of Stability Tests

Recrystallization of desloratadine (10 g) from iso-propanol (25 ml) at 80° C. The cooling term was regulated. The temperature was cooled to 0° C. in 30 min, which generated a mixture. The X-ray Powder Diffraction measurement showed that the ratio of the two polymorphic forms was 74:26 (Form 1/Form 2).

| | | 25° C., 60% RH | | 40° C., 75% RH | |
|---|---|---|---|---|---|
| | Time | Form 1 | Form 2 | Form 1 | Form 2 |
| Time | (weeks) | (%) | (%) | (%) | (%) |
| 0 week | 0 | 74 | 26 | 74 | 26 |
| 1 week | 1 | 79 | 21 | 77 | 23 |
| 2 weeks | 2 | 74 | 26 | 81 | 19 |
| 1 month | 4 | 79 | 21 | 78 | 22 |
| 2 months | 8 | 75 | 25 | 78 | 22 |
| 3 months | 12 | 74 | 26 | 75 | 25 |
| 6 months | 24 | 77 | 23 | 75 | 25 |

### Example 4 of Stability Tests

Recrystallization of desloratadine (10 g) from iso-propanol (25 ml) at 80° C. The solution was seeded with Form II. The cooling term was regulated. The temperature was allowed to 0° C. in 30 minutes. The X-ray Powder Diffraction measurement showed that the ratio of the two polymorphic forms was 76:24 (Form 1/Form 2).

| | | 25° C., 60% RH | | 40° C., 75% RH | |
|---|---|---|---|---|---|
| | Time | Form 1 | Form 2 | Form 1 | Form 2 |
| Time | (weeks) | (%) | (%) | (%) | (%) |
| 0 week | 0 | 76 | 24 | 76 | 24 |
| 1 week | 1 | 87 | 13 | 76 | 24 |
| 2 weeks | 2 | 80 | 20 | 82 | 18 |
| 1 month | 4 | 82 | 18 | 88 | 12 |
| 2 months | 8 | 86 | 14 | 85 | 15 |
| 3 months | 12 | 81 | 19 | 82 | 18 |
| 6 months | 24 | 84 | 16 | 82 | 18 |

Analytical HS GC Method for Determination of Residual Solvents of Desloratadine

1. GC Parameters (Agilent 6890 N)

| Column: | | Restek Stabilwax 10609 |
|---|---|---|
| | | (30 m * 0.32 mm I.D., 0.1 μm film) |
| Carrier gas: | | Helium |
| Detector: | | FID |
| Head pressure (constant): | | 100.0 kPa |
| Nominal initial flow: | | 3.4 ml/min |
| Split ratio: | | 10:1 |
| Inlet temperature: | | 140° C. |
| Detector temperature: | | 250° C. |
| Temperature program: | | |
| Level 1: | Initial temperature: | 50° C. |
| | Initial time: | 2.0 min |
| Level 2: | Rate: | 20° C./min |
| | Final temperature: | 70° C. |
| | Final time: | 0.0 min |
| Level 3: | Rate: | 50° C./min |
| | Final temperature: | 200° C. |
| | Final time: | 0.4 min |

| | |
|---|---|
| Typical retention times: | |
| n-Heptane and homologues | 1.3 min |
| Ethanol | 1.6 min |
| i-Butyl acetate | 2.0 min |
| Toluene | 2.1 min |
| i-Butanol | 2.4 min |

2. Headspace Parameters (Agilent 7694)

| | |
|---|---|
| Matrix: | Dimethyl Sulfoxide (DMSO) |
| Temperature values: | |
| Sample oven: | 80° C. |
| Sample valve: | 100° C. |
| Transfer line: | 110° C. |
| Time values: | |
| GC cycle time: | 13.0 min |
| Sample equilibration time: | 15.0 min |
| Vial pressurization: | 0.20 min |
| Loop fill: | 0.10 min |
| Loop equilibration time: | 0.10 min |
| Inject of sample: | 0.10 min |
| Shaking agitation: | Low |
| Loop volume: | 1.0 ml |
| Vial pressure: | 30.0 kPa |

[image]

3. Solutions
Standard Stock Solution

Transfer about 10 ml DMSO into a 25.0 ml volumetric flask, than weigh into this flask accurately about 87.5 mg of n-Heptane, 50.0 mg of Ethanol, 100.0 mg of i-Butyl acetate, 22.0 mg of Toluene and 12.5 mg i-Butanol, fill it up to volume with DMSO and homogenize.

Standard Solution

Transfer 1.0 ml of stock solution into a 10.0 ml volumetric flask, fill it up with DMSO to volume and homogenize. Pipet 1.0 ml of this solution into a 20.0 ml headspace vial than close it.

(c_{n-Heptane}≈350 μg/ml, c_{Ethanol}≈200 μg/ml, c_{iButyl acetate}≈400 μg/ml, c_{Toluene}≈88 μg/ml, c_{i-Butanol}≈50 μg/ml)

Sample Solution

Dissolve about 100.0 mg of Desloratadine sample accurately weighed in 1.0 ml of DMSO in a 20.0 ml headspace vial, than close it.

4. System Suitability Requirements

Peak resolution (Rs) ≧2.5 between the i-Butyl acetate and the Toluene peaks in the standard chromatogram.
5. Evaluation

$$Solvent content [ \% ] = \frac{{A}_{sample} * {c}_{std} * 100}{{c}_{sample} * {A}_{std}} , where$$

Aₛₐₘₚₗₑ area of the relevant solvent in the sample chromatogram
c_{std} concentration of the relevant solvent in the standard solution [mg/ml]
cₛₐₘₚₗₑ concentration of Desloratadine in the sample solution [mg/ml]
A_{std} area of the relevant solvent in the standard chromatogram.

Having thus described the invention with reference to particular preferred embodiments and illustrative examples, those in the art would appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used as a guidance. All references mentioned herein are incorporated in their entirety.

What is claimed is:

1. A process for preparing a mixture of crystalline Form I and Form II of desloratadine comprising combining a solution of desloratadine in an organic solvent selected from the group consisting of a C₂ to C₅ alkyl acetate, butanol, isobutanol, toluene and chloroform with a C₅ to C₁₂ hydrocarbon to precipitate the mixture, recovering the mixture, wherein before combining, the solution has a temperature that is at least about 40° C. higher than the hydrocarbon.

2. The process of claim 1, wherein the combining is carried out in less than about 10 minutes

3. The process of claim 1, wherein the temperature of the hydrocarbon is at about −10° C. to about 20° C. and the temperature of the solution is about 60° C. to about 100° C.

4. The process of claim 3, wherein the temperature of the solution is about 90° C.

5. The process of claim 3, wherein the temperature of the hydrocarbon is about 0° C.

6. The process of claim 1, wherein the organic solvent is iso-butyl acetate.

7. The process of claim 1, wherein the hydrocarbon is n-heptane.

8. The process of claim 1, wherein the solution has a temperature that is at least about 60° C. higher than the hydrocarbon.

9. The process of claim 1, wherein the mixture is recovered by filtration.

10. The process of claim 1, further comprising drying the mixture.

11. The process of claim 1, wherein the mixture has a residual level of any one of the organic solvent and C₅ to C₁₂ hydrocarbon that is less than about 5000 ppm.

12. The process of claim 11, wherein the residual level of any one of the organic solvent and C₅ to C₁₂ hydrocarbon is of about 50 ppm to about 4000 ppm.

13. The process of claim 1, wherein the mixture comprises about 35-82% desloratadine Form I and about 18-65% desloratadine Form II.

14. The process of claim 13, wherein the mixture comprises about 55-82% desloratadine Form I and about 18-45% desloratadine Form II.

15. The process of claim 1, wherein at least about 0.5 Kg of the mixture is recovered.

16. The process of claim 15, wherein at least about 1 Kg of the mixture is recovered.

17. The process of claim 16, wherein at least about 10 Kg of the mixture is recovered.

18. The process of claim 1, wherein the desloratadine combined with the organic solvent is obtained by the process comprising:
a) combining a solution of a desloratadine salt in an organic solvent selected from a C₂ to C₅ alkyl acetate, butanol, isobutanol, toluene and chloroform and an aquoues base to obtain an organic phase containing desloratadine and an aqueous phase;
b) separating the organic phase from the aqueous phase;
c) recovering desloratadine from the organic phase.

19. The process of claim 18, wherein the salt is the acetate salt.

20. The process of claim 18, wherein the organic solvent is iso-butyl acetate.
